SEQUENCE LISTING (29 sequences)

SEQ ID NO: 1 - protein, 353 aa, Mus musculus
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 2 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 3 - DNA, 5236 bp, Artificial Sequence
  5′ homology arm
acactcaaccttgttgtcttgtggttgtatcttcaatgatatgtggcaatatccctataaacagcactttccctgggctgatctgtcagaccttctgtaacttatgtgggaaattaaactgattagatggcaaagacttttctgataggttcgttctactattgataatacaagagcttttagttaaattttatttatttatctctgtatgagcttttgttaaatacaggtttccatgtgcaaactcttgtaagcaaatgctaggagatttgtagttggaagtgagagtgttactgtaaataacaaacagaggtctactccaaagaaaagcagtgttgatttgaaaagagtgcaatgttacaacgtgcagcgagattcacagtaaccaccagtttatacaatgagcatggagcatggcaaatccagaaacacaaaaatgaaggttgagtaagatactgagaaacagtatccctggcctcgaggattaatccaaatgttgccagtctgaggctgaggccatagtggagggaaagctgtcccagagatgttttctttgtaaggttgtgggggcattgtgtggggtgtggtttggacagtggtttgttagcaaggtttattttatttaaggctacagagacaaggcggggattaagaaggctgttgctcttgcagaggccccaaggttaattcccagcactcacatggtggctcataactccagcttcagggcatcctatgccctctactggcttctgtgggcactgcatacatgtgacatttactctcgtacacaaatgtagagataaaaataaaactgcatcccttattaaaagagtttatttgcgtgtgatgcgcacccatgcgcgcgtgtttgcttatctgagtggtcctgtagaggtcagtagaggcaggatgcatcagtctctgcctcctcctgaaacagaggctctcacagaactcatgatttctgtgatcggctggactctagcaaacctagaaccctgtcatctttgctcttcttagagctggggttacaagcttttgcaggaacgtcggcttgttacacaggtgctgggaaccaaattctggtcctcatgatttataagggagtactcttcactgctgagccgttctttctatctgtggcagaagttctgtgtcagtgcctttgggagtatgtgagaacccttccaatgcaacctcatggttcatttgtttgttcctcctttcttggttcattcattcattaattcttttttttccccccatccttcttcctcaggtggttctaacatgccttgctttcaaagatttaaagattttaaagatttatttcgtgtgaacaggtcttttgcctgagtgtctgtctgtgcaccacatgcgtgctgggtactggagaaggccagaagagggcatagcatctcctaaactggatttaaagccagttgtgtgttgccagccacgtgggggctgggaatgaacccaggtgctttggaagcagagcagccggtgacactcttacttgtggagccactctccggctcctttatgtcacatttgggtcacacaagccactccatctcagttctgagaattttcataacagataatcctaaaagagttttttctaatgtcatctgtcttttagttttaatgcaggttaataagaggggccccctgagctgtgcttcaggaagcttcagtgtaggctctggccttaccaccaaatagctgtgtgtgtgtgtgtgtgtatgtgtgtgtgtgtgtgtgtgtgtgtgtgtatgagagcgtgtgtgtgtgtactcgagtgtgtgtgtgtgtgtgtgtgagagagagagagagagagagagagagagtgtgtgactgtgtgtgtgtatgtgtgtgactgtgtgattgtgtgtgtgtgcacgcccttgcccctgtgtttctgtatgtgtgtactttgcctgcccagtctctccccattgtgctgtgatactggagtgattcatttcacgtccatggaactaaacattcagaacgaaagaggttgagtgtgagagaaagacagactggcaggctgtcttagttagggttttattgctgtgaacagacaccatgaccaaggcaactcttataaggacaacacttagtaggggctggcttactgggtcagaggttcagtccattatcatcatggtgggaagcatggcagcatccaggcaggcatggtgcatgagctgagatttctacatcttcatctgaaggctgctagtggaagactcacttccaggcagctaggatgagggccttaaagcccacactcacagtgaatgacacacctactccaacaaggccacacctctcaatagtaccactccctgggccaagcatataaaaaccatcacaaagtcatggatagatcccaagacagtaattccagtattcaggaggcagagtcaggcagacctctaagtgctaggccagcctagtctacatagggaagtccaggctagcttaatctacacagaggggactaacacacagggagaggacagatttgtgagtggccctgctgggcaggggtggcagccagttgtctctgacctggcacatcaccgatggagttggattcatactttccattaccccgacgagcatcagcagcacctgagagtccgctgttcacagtcagctcggcactgacattttcttatcgctattctgtcttaaaggccggggcttgccccaagctctgttaaaacctcttggtgaggagaatcataggcatgatctgaggtgtcagagatgcttctaaatgataaactcactcgttgtcagaagacagattcacgggtcacagagcagaagctgagaggaacatgctgtcacctgaatagtgagtgtccaagtgccgaggccagagaggggaacacatgcctgcagctctagttcccagggaggcagagttatgtggctccctggggctcactggtaaccaacctagcccatttcaggagacccaaactagagagagaccacatcaaaacaacagaagcaaatggtaagagacactaagagcaaacgttctgctgagcagcagtggtgtacgcctttaatcccagtattccagaggcctaggcaggtggctctcggagttcaaggccagcctgctctacagaatgagttctaagacagccactgttctgcagagaaaccctgttttgaacaacaaaacaaacaaacaagcaaacaaacaaacaggaactaagagtagatggacagattccctcccataccaccatccagaggaaaggtgatgccccctgcttatctctgccccagatctgaggtgaaaagacaggaagaaggtactgagagtcaaggagccctggttgcatagcacgctgttccattgctctggagacagggtgggtctgtcccaaacagcagaggggttggggctgtaagtcagaggtagctcttactgttccaacctttggattggctgaggcatggcatatagctgaagtgctcaggaggaggagtcagcacagcaagcagacatgtggcagaggagtgggcagctctgaaacctcagaagaaaggctcgctcagataattggtaagaatcatccttggggttagggctcagaggcagtgcgggcaccgttttcatctccttacagagagatcaggagaaggctgatatttcgggatcataagagttttgagtgtctccaaagcctgagcccacaagcgcgtgtgtctcttggtggctgataggagcatgtcccttgttctccaggacacagaaagggctgaaagctctcacaggagtaaaaaaattagacaagacagcattttctggcgatcatcctttctccacagaaaacttccattcaatcccacctccttgtatgtctgtcaatggagtcttagactctgcattgtgtggggtgtggggaacgggacctaggacctggctcgggtcctaagcatgtgctgtgcccccaggccctgtaatgttttatttcgtggtgtgtgtgtgtgtgtgtgtgtgtgtgtgtgtgtgagaatatgtgagtgtgcgcatgtacaggaggtcagaggccagccttggtgctatgtttcaggcgctctcccctcgctttgtgctcaccaagcaggccaggctggccagggccacggagctctgcctgtctctgcctcccctgcgtgggcattgcgagcacacactgccacgcatgggtttaggggtttgaactgaggtctccatggttgcttagcaagcacctcgcttacggagatatcccctcagccccagtgtttgacacgactgaattccagcctttaccaggcttacctctggcgaacccggtgcttctcgttttgagaggaattgggatcatttttttttcctattgcactttaaatcgttttgaggggggggggttatggaagcaatgagtgaatgaactaaattcaagaatatcataggatacatccgaagcaaaatacaaaatgagacatcatatatgcagcataggaatgagtaagcaaatgttgtctgttgaatacatatatactttgtttgtttgagacagggcttctctgtgtagccctggctgtcctggaacccactctgtagaccaagctagcctcaaactcagagagccaccctcttctgcctcccaagtgctgggactaaagatgtgtatcaacacacctggcctgtattttgtttgtggatccataaaatgaaagagaaaccaggcagagtggcgcgtgcttttcatcccagcacctgggaagcagaggcaggtgaatctctgggagtttgaggtcagcttggtctatatagtaagttccaggacaactagggctatatagtgagactatctaaaaataccaaaattgactaattacaatgggggggtgggggagagagagagagagagagaggccaagcaaggtggtacagatctgtaatacctccactgagaaggctgaggcaggaggatcatgagtagtctgggctatgtagcaagttccataccagcccaaaccacttagtgagaccttgtatgaaggaagcaaggtctagggagaacagcttgatgggagagcgcttgccgcacactaacaccctgacattgagccgcagggagaaaggggaggtgtttaagggcttcaatctgtgaatacagccctcagcacctctcacgtgcctgcttgaccaggtcttcctgcctcg

SEQ ID NO: 4 - DNA, 3851 bp, Artificial Sequence
  3′ homology arm
ctgagaactggacccaggggctggagagatggctcagtggttaagagcaccgactgctcttccgaaggtcctgagttcaaatcccagcaaccacatggtggctcacaaccatccataatgagatctgacgccctcttctggtgcgtctgaagatagctacagcgtacttagatataataataaataaatcttaggaaaaaaaaaaagaaaaaaaaaaagagaactggacccagttctcagcacccacatggtggttcaaaccatctgcaactccagtttcaggggatctggcgtccccgtctggttctgtggactctaggaacacatatagggagcatacataactgcaggcaaaacacttatacacatagaatagagtaaaataaaatctattctctgaaatggtaaaaaaaaaaaggtatttgtgtgaaaaaatggcattacttgctctttgaaggtttgatacaacttattaataaaccaatccagggtacttctgtacaaagatttgcatttaattaccaaatggaagcgttcagcctttttatctgcttgtggtttgtaagaaattctgctattctccttaagttcacaagtacatgagtacgaattcttgacaattaattttgttgaacaatctttttgtagtatgagggtgtccctttctggtcctgatactgatcattgtggctcccccccccacccccgtctcctcctcccctccctccctcccctctcctagatcagttttgtaggacttatctgttgcttaaatctctgcatcgagccagcctttggcctcgctgacttcctgtctcctctgctcgttgatttctgtttttattttcatttcacctatttgctttgagcctggtttttatatgggctcctggaatcgtctattgtctcagtattcacttggggtgcattttacacattttaagtttggtcttagccagaagtctgagaagtagtcgtctactcctgccttagccactgttctactgctgtgaagagacgccgtggctaaagcaacttatagaagaagacatttaactgggggcttgttcaaagcttcagagggtttatcaccatggcggggagcagggtggcaggcagggaggcatggtgagagaagcagctaggagctttccatcctgatccacagccaggagagagagagagagagagagagagagagagagagagagagacagagacagagacagagacacagaggagagacacagagagacagagagacagagagagacagagagacagcgcttcttcaacaaggcaacacaccttctaatccttttcaacacttcaccaccaactgggggctaagtatgcaaatatatgagcctatggggctcacttaaaccaccacaaccagcagaaatgttattatatcaagaattcaaattattctggaaagtaaagaagaattcataacattgagaagaaaaaaaccacgacaacttcatacagtatttacttacactcttctcagagaacatacacatagggttttattcctttaagatatatcaagaattattttatgatgcaagacatggtttccattcacccgtgaaggtaacaggttgcttctgcaggtagctagtcacgcagttcgaggcttctgtgttctcgctctttttctgatttttttttcctttctgcagctatcttgttgcttttgtgaatcggggagtttaatgagggttgctttcagaggtctgtgggtgaagagcgttttgcaggaacaagagcaactaccactggtcacatcactgaagaaaatgtctgcccactaagacttttgtactctttatttgtttaaaacaacttttatttttatttttatttttatttattttttatttttggttttttgagacaaggtttctctgtatagccctggctgtcctggaactcactttgttgaccaagctggcctcgaactcccaagtactgggactaaaagtgtgtgccgccaccacccggcacaactgttatcttattttttttttttaaatgtgatttattatgagtgtagagatcagatcagaggacaacttgcaagagttggccctgtcttcccagcgcatgggtctcagggatggagtccaggcaccttcccctttgagcggctgcacaggctgaatccttggtgatttccccctggttgtttatcccatgagtcgataggagaggcactgcaaaacacccaactctgattatggcttatttattttccattttaattctgtttgtttttactgtatgttttgaagctgtttttttttttaagatttttttatttatatgagtactttgtagctgtcttcagacacaccagaagaaggcatcggatcccactacagatggttgtgagccaccatgtggttgctgggaattgaactcaggacctctggaagagcagttggtgttcttaaccactgagccatctctccagccactgaagctgttattagtatggacaaataaagaattgttctgcatgtttttaaaccaaatagcctcttttgttattatgaaatttcctttgtgttctttttgagaggtaagtaggtgggtttgagacaggatctatctatatagccctagctatcctggaactcactgtgtagtccaggctagccttgaactcacagagctccacatacctctgccttcagagtgctaggattaaaagcacgagtcaccatgtctggccgaataaatatttctacaaagcaagtttgaggttagcctaggatacagaagaccctgttcccccccacacaaaaaaaaagtttaaggaaagataaaattatttattgttatggcaagaggttgccaagttggaatcttagagtaggatgttgccgaagccacaggctcatagctcatcagttgtatcagctagcaaggccaccatcttcctcacagtctccatgcttctctgactgaaccttgcaagcacccagtgggcaacatgctgtgtggggtgggacaggcaactctgcagtgtgtgtgagacatggggagtggagaggggcttcctccgacatttttcaagactgtcagcttccaggagtcctgctcagatggttcttatttagcaagagtcactcgggcatcttgagtcacactaagctttctgatggggcaaggacctgacttggtccagctgcatcccttacagctggcctcaggtaagctctctgaactccaagaggccgtgggtaagagggggtggagtgagcggagacagagggaccatctgtcctgcctgcccacacagctgcctgatatccttacagaacccgtcaactcccttcatacatttaaacgaaaaatcttagtaacgtgatgaaataaacagcggacaggttaggacttgtttgtttgtttgtgcgtttgtttgttttcttttggttatcacacttggggtaactgatataactgtttgctgcttccaattccttacccattaaaggggactgggaatgaagctagaaccgggtgaggaattggttagaaccaggaagttgtgctctatcgccacctgctgttcactgtctagcggtaccaccagctgcccacagttgcaaagaactggaattccctgatgctttgggttcatctggcaaacccgcatttggactatgtgattgaatgcacattaacaagcaagcaggtaaacacttgcaacagtggggtacagttgggatcccctgcctgtcttgctcattgccacatcccgggtcccagctgttgtgtacttgctgggtggattgagaagctagcccctgaagatcatgagaaccagggagaac

SEQ ID NO: 5 - DNA, 1068 bp, Artificial Sequence
  the nucleotide 39332332-39333399 of NCBI accession number NC_000003.12
atggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtga

SEQ ID NO: 6 - DNA, 80 bp, Artificial Sequence
  connection between the human CCR8 sequence upstream of the A fragment and the mouse CCR8 gene
acctctcacgtgcctgcttgaccaggtcttcctgcctcgatggattatacacttgacctcagtgtgacaacagtgaccga

SEQ ID NO: 7 - DNA, 81 bp, Artificial Sequence
  connection between the downstream of the human CCR8 sequence and the mouse CCR8 gene
ctcctcccgttcctccagcgtagactacattttgtgaggggagtgtgcagggcaggcagactccacctgcattgcccttcc

SEQ ID NO: 8 - DNA, 88 bp, Artificial Sequence
  connection between the 5′ end of the Neo cassette and the mouse gene
agatggctctgcagctaaaggcacttgttcttactaagcttgatatcgaattccgaagttcctattctctagaaagtataggaacttc

SEQ ID NO: 9 - DNA, 113 bp, Artificial Sequence
  connection between the 3′ end of the Neo cassette and the mouse gene
gaagttcctattctctagaaagtataggaacttcatcagtcaggtacataattaggtggatccactagttctagactgagaactggacccaggggctggagagatggctcagt

SEQ ID NO: 10 - DNA, 1162 bp, Artificial Sequence
  mRNA sequence of the humanized mouse CCR8
tggcagaggagtgggcagctctgaaacctcagaagaaaggctcgctcagataattggtcttcctgcctcgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggggagtgtgcagggcaggcagac

SEQ ID NO: 11 - DNA, 24 bp, Artificial Sequence
  primer
tgctagtggaagacagacttccag

SEQ ID NO: 12 - DNA, 24 bp, Artificial Sequence
  primer
cagcaaggagcaacttgccatttg

SEQ ID NO: 13 - DNA, 20 bp, Artificial Sequence
  primer
gctcgactagagcttgcgga

SEQ ID NO: 14 - DNA, 25 bp, Artificial Sequence
  primer
ctcacgagccatctgaaaccctgtt

SEQ ID NO: 15 - DNA, 25 bp, Artificial Sequence
  primer
ctgctctcacaccatcctgactgag

SEQ ID NO: 16 - DNA, 27 bp, Artificial Sequence
  primer
tctcactgaacttagagccatctgtga

SEQ ID NO: 17 - DNA, 27 bp, Artificial Sequence
  primer
ctcagccagtcacccatttcaaaaagc

SEQ ID NO: 18 - DNA, 26 bp, Artificial Sequence
  primer
gtctccaccgtgtcctctacatcctg

SEQ ID NO: 19 - DNA, 21 bp, Artificial Sequence
  primer
ggatcggccattgaacaagat

SEQ ID NO: 20 - DNA, 22 bp, Artificial Sequence
  primer
cagaagaactcgtcaagaaggc

SEQ ID NO: 21 - DNA, 25 bp, Artificial Sequence
  primer
acactaacaccctgacattgagccg

SEQ ID NO: 22 - DNA, 22 bp, Artificial Sequence
  primer
gatggtggctgtgacagcagcc

SEQ ID NO: 23 - DNA, 24 bp, Artificial Sequence
  primer
cagcaaggagcaacttgccatttg

SEQ ID NO: 24 - DNA, 23 bp, Artificial Sequence
  primer
tcagcctcctgaacgctggacca

SEQ ID NO: 25 - DNA, 22 bp, Artificial Sequence
  primer
tagctatcttcagacgcaccag

SEQ ID NO: 26 - DNA, 25 bp, Artificial Sequence
  primer
gacaagcgttagtaggcacatatac

SEQ ID NO: 27 - DNA, 24 bp, Artificial Sequence
  primer
gctccaatttcccacaacattagt

SEQ ID NO: 28 - DNA, 1484 bp, Homo sapiens
gtagtgggaggatacctccagagaggctgctgctcattgagctgcactcacatgaggatacagactttgtgaagaaggaattggcaacactgaaacctccagaacaaaggctgtcactaaggtcccgctgccttgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggatcaatgaagactaaatataaaaaacattttcttgaatggcatgctagtagcagtgagcaaaggtgtgggtgtgaaaggtttccaaaaaaagttcagcatgaaggatgccatatatgttgttgccaacacttggaacacaatgactaaagacatagttgtgcatgcctggcacaacatcaagcctgtgattgtgtttattgatgatgttgaacaagtggtaactttaaaggattctgtatgccaagtgaaaaaaaaagatgtctgacctccttacatat

SEQ ID NO: 29 - DNA, 1156 bp, Mus musculus
tggcagaggagtgggcagctctgaaacctcagaagaaaggctcgctcagataattggtcttcctgcctcgatggattacacgatggagcccaacgtcacgatgaccgactactaccctgatttcttcaccgccccctgtgacgcagagttcctcctcaggggcagcatgctgtatctggccatcttgtactgcgtcttgtttgtgctgggccttctggggaacagcctggtcatcttagtcctcgtgggctgcaagaaactgaggagcatcacagatatctacctcctgaacctggccgcatccgacctgctctttgtcctctctattccttttcagacccacaacctgctggaccagtgggtgtttgggactgcgatgtgtaaggtggtctctggcctttattacattggttttttcagcagtatgttcttcatcaccctaatgagtgtggacaggtatctggctattgtccacgctgtctatgccatcaaggtgaggacggccagcgtgggcacagccctgagtctgacagtgtggctggctgctgtcacagccaccatccccttgatggttttttaccaagtggcctctgaagacggcatgctacaatgtttccagttttatgaagagcagtctttgaggtggaagctctttacccactttgaaatcaacgccttgggtctgctgctcccctttgccatcctcctgttctgctatgtcaggatcctgcagcagctgcggggctgcctgaaccacaacaggaccagagccatcaagctggtgctcaccgtagtcattgtgtctttactcttctgggtcccattcaacgtggcccttttcctcacgtccctgcacgacctgcacatcttggatggatgtgccacgaggcagaggctggctctggccatccatgtcacagaggtcatctcttttacccactgctgcgtgaaccccgtcatctacgcgttcataggagagaagtttaagaaacacctcatggatgtgtttcaaaagagctgcagccacatcttcctctacttagggagacaaatgcccgtgggggcgttggaaaggcagctgtcctcgaaccagcgatcttcccattcttccaccctggatgacatcttgtaaggggagtgtgcagggcaggcagac

(19) United States
(12) Patent Application Publication
(10) Pub. No.: US 2023/0320332 A1
(43) Pub. Date: Oct. 12, 2023

(54) GENETICALLY MODIFIED NON-HUMAN ANIMAL WITH HUMAN OR CHIMERIC CCR8

(71) Applicant: BIOCYTOGEN PHARMACEUTICALS (BEIJING) CO., LTD., Beijing (CN)

(72) Inventors: Yuelei SHEN, Beijing (CN); Yang BAI, Beijing (CN); Chaoshe GUO, Beijing (CN); Meiling ZHANG, Beijing (CN); Rui HUANG, Beijing (CN); Chengzhang SHANG, Beijing (CN)

(21) Appl. No.: 18/025,302

(22) PCT Filed: Sep. 10, 2021
(86) PCT No.: PCT/CN2021/117825

§ 371 Date: Mar. 8, 2023

(30) Foreign Application Priority Data
Sep. 11, 2020 (CN) 202010953820.8
Mar. 4, 2021 (CN) 202110238621.3
Mar. 12, 2021 (CN) 202110270555.8

(51) Int. Cl. A01K 67/027; C07K 14/715; C12N 15/85; A61K 49/00

(52) U.S. Cl. CPC A01K 67/0275; C07K 14/7158; C12N 15/8509; A01K 67/0278; A61K 49/0008; A01K 2227/105; A01K 2207/15; A01K 2217/072; A01K 2267/0331; C12N 2800/107

(57) ABSTRACT

Provided are genetically modified non-human animals that express a human or chimeric (e.g., humanized) CCR8, and methods of use thereof.

### CLAIM OF PRIORITY

This application claims the benefit of Chinese Patent Application App. No. 202010953820.8, filed on Sep. 11, 2020, Chinese Patent Application App. No. 202110238621.3, filed on Mar. 4, 2021, and Chinese Patent Application App. No. 202110270555.8, filed on Mar. 12, 2021. The entire contents of the foregoing are incorporated herein by reference.

### TECHNICAL FIELD

This disclosure relates to genetically modified animal expressing human or chimeric (e.g., humanized) CCR8, and methods of use thereof.

### BACKGROUND

The immune system has developed multiple mechanisms to prevent deleterious activation of immune cells. One such mechanism is the intricate balance between positive and negative costimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

This disclosure relates to genetically modified animal expressing human or chimeric (e.g., humanized) CCR8, and methods of use thereof.

In one aspect, the disclosure relates to a genetically-modified, non-human animal whose genome comprises at least one chromosome comprising a sequence encoding a human or chimeric CCR8.

In some embodiments, the sequence encoding the human or chimeric CCR8 is operably linked to an endogenous regulatory element at the endogenous CCR8 gene locus in the at least one chromosome.

In some embodiments, the sequence encoding the human or chimeric CCR8 comprises a sequence encoding an amino acid sequence that is at least 70%, 75%, 80%, 85%, 90%, 95%, 99%, or 100% identical to SEQ ID NO: 2.

In some embodiments, the sequence encoding the human or chimeric CCR8 is operably linked to an endogenous 3′-UTR (e.g., immediately after 3′-UTR).

In some embodiments, the animal is a mammal, e.g., a monkey, a rodent or a mouse.

In some embodiments, the animal is a mouse or a rat.

In some embodiments, the animal does not express endogenous CCR8 or expresses a decreased level of endogenous CCR8 as compared to that of an animal without genetic modification.

In some embodiments, the animal has one or more cells expressing human or chimeric CCR8.

In some embodiments, the animal has one or more cells expressing human or chimeric CCR8, and human CCL1, human CCL8, human CCL16 or human CCL18 can bind to the expressed human or chimeric CCR8.

In some embodiments, the animal has one or more cells expressing human or chimeric CCR8, and endogenous CCL1, endogenous CCL8, endogenous CCL16 or endogenous CCL18 can bind to the expressed human or chimeric CCR8.

In one aspect, the disclosure relates to a genetically-modified, non-human animal, whose genome comprises a replacement of a sequence encoding a region of endogenous CCR8 with a sequence encoding a corresponding region of human CCR8 at an endogenous CCR8 gene locus.

In some embodiments, the sequence after the replacement is operably linked to an endogenous regulatory element at the endogenous CCR8 locus, and one or more cells of the animal express human CCR8 or chimeric CCR8.

In some embodiments, the animal does not express endogenous CCR8 or express a reduced amount of CCR8 expresses a decreased level of endogenous CCR8 as compared to that of an animal without genetic modification.

In some embodiments, the replaced region is located immediately after 5′-UTR at the endogenous CCR8 locus.

In some embodiments, the animal has one or more cells expressing a chimeric CCR8 having one or more humanized extracellular regions, transmembrane regions, and cytoplasmic regions.

In some embodiments, one or more of the humanized extracellular regions comprise a sequence that is at least 50%, 60%, 70%, 80%, 90%, 95%, or 99% identical to the corresponding extracellular region of human CCR8.

In some embodiments, one or more of the humanized extracellular regions of the chimeric CCR8 has a sequence that has at least 10, 20, 30, 40, 50, 60, 70, 80, 90, or 95 contiguous amino acids that are identical to a contiguous sequence present in the corresponding extracellular region of human CCR8.

In some embodiments, the animal is a mouse, and the replaced region comprises or consists of the entirety or a portion of the coding sequence in exon 2 of the endogenous mouse CCR8 gene.

In some embodiments, the animal is heterozygous with respect to the replacement at the endogenous CCR8 gene locus.

In some embodiments, the animal is homozygous with respect to the replacement at the endogenous CCR8 gene locus.

In one aspect, the disclosure relates to a method for making a genetically-modified, non-human animal, comprising: replacing in at least one cell of the animal, at an endogenous CCR8 gene locus, a sequence encoding a region of an endogenous CCR8 with a sequence encoding a corresponding region of human CCR8 gene.

In some embodiments, the sequence encoding the corresponding region of human CCR8 gene comprises exon 2, or a part thereof, of a human CCR8 gene.

In some embodiments, the sequence encoding a corresponding region of human CCR8 gene encodes a sequence that is at least 90% identical to SEQ ID NO: 2.

In some embodiments, the animal is a mouse, and the endogenous CCR8 locus is exon 2 of the mouse CCR8 gene.

In some embodiments, the replaced region is located in exon 2 of the mouse CCR8 gene.

In one aspect, the disclosure relates to a non-human animal comprising at least one cell comprising a nucleotide sequence encoding a humanized CCR8 polypeptide, which comprises at least 50 contiguous amino acid residues that are identical to the corresponding contiguous amino acid sequence of a human CCR8. In some embodiments, the animal expresses the humanized CCR8.

In some embodiments, the humanized CCR8 polypeptide has at least 50 contiguous amino acid residues that are identical to the corresponding contiguous amino acid sequence of a human CCR8 extracellular region.

In some embodiments, the humanized CCR8 polypeptide comprises a sequence that is at least 90%, 95%, or 99% identical to SEQ ID NO: 2.

In some embodiments, the nucleotide sequence is operably linked to an endogenous CCR8 regulatory element of the animal (e.g., 5′-UTR).

In some embodiments, the humanized CCR8 polypeptide comprises one or more humanized extracellular region, one or more humanized CCR8 transmembrane region and/or one or more humanized CCR8 cytoplasmic region.

In some embodiments, the nucleotide sequence is integrated to an endogenous CCR8 gene locus of the animal.

In one aspect, the disclosure relates to a method of making a genetically-modified mouse cell that expresses a human CCR8 or a chimeric CCR8, the method comprising: replacing at an endogenous mouse CCR8 gene locus, a nucleotide sequence encoding a region of mouse CCR8 with a nucleotide sequence encoding a corresponding region of human CCR8, thereby generating a genetically-modified mouse cell that includes a nucleotide sequence that encodes the human CCR8 or the chimeric CCR8. In some embodiments, the mouse cell expresses the human CCR8 or the chimeric CCR8.

In some embodiments, the entire coding sequence of mouse CCR8 gene is replaced by the entire coding sequence of human CCR8 gene.

In some embodiments, the chimeric CCR8 comprises: one or more of the extracellular regions of human CCR8; and one or more of the transmembrane regions; and/or one or more of the cytoplasmic regions of mouse CCR8.

In some embodiments, the animal further comprises a sequence encoding an additional human or chimeric protein.

In some embodiments, the additional human or chimeric protein is cytotoxic T-lymphocyte-associated protein 4 (CTLA-4), Lymphocyte Activating 3 (LAG-3), B And T Lymphocyte Associated (BTLA), Programmed cell death protein 1 (PD-1), Programmed death-ligand 1 (PD-L1), CD27, CD28, CD40, CD47, CD137, CD154, T-Cell Immunoreceptor with Ig And ITIM Domains (TIGIT), T-cell Immunoglobulin and Mucin-Domain Containing-3 (TIM-3), Glucocorticoid-Induced TNFR-Related Protein (GITR), or TNF Receptor Superfamily Member 4 (OX40).

In some embodiments, the animal or mouse further comprises a sequence encoding an additional human or chimeric protein.

In some embodiments, the additional human or chimeric protein is CTLA-4, LAG-3, BTLA, PD-1, PD-L1, CD27, CD28, CD40, CD47, CD137, CD154, TIGIT, TIM-3, GITR, or OX40.

In some embodiments, the animal has a tumor.

In one aspect, the disclosure relates to a method of determining effectiveness of an anti-CCR8 antibody for the treatment of cancer, comprising: administering the anti-CCR8 antibody to the animal described herein having a tumor; and determining the inhibitory effects of the anti-CCR8 antibody to the tumor.

In some embodiments, the tumor comprises one or more cells that express a CCR8 ligand.

In some embodiments, the tumor comprises one or more cancer cells that are injected into the animal.

In some embodiments, determining the inhibitory effects of the anti-CCR8 antibody to the tumor comprises measuring the tumor volume in the animal.

In some embodiments, the tumor cells are breast cancer cells, colon cancer cells, or lung cancer cells.

In one aspect, the disclosure relates to a method of determining effectiveness of an anti-CCR8 antibody and an additional therapeutic agent for the treatment of a tumor, comprising: administering the anti-CCR8 antibody and the additional therapeutic agent to the animal described herein having a tumor; and determining the inhibitory effects on the tumor.

In some embodiments, the animal further comprises a sequence encoding a human or chimeric PD-1.

In some embodiments, the additional therapeutic agent is an anti-PD-1 antibody.

In some embodiments, the tumor comprises one or more tumor cells that express PD-L1.

In some embodiments, the tumor is caused by injection of one or more cancer cells into the animal.

In some embodiments, determining the inhibitory effects of the treatment involves measuring the tumor volume in the animal.

In some embodiments, the animal has breast cancer, colon cancer, or lung cancer.

In one aspect, the disclosure relates to a protein comprising an amino acid sequence, wherein the amino acid sequence is one of the following: (a) an amino acid sequence set forth in SEQ ID NO: 2; (b) an amino acid sequence that is at least 90% identical to SEQ ID NO: 2; (c) an amino acid sequence that is at least 91%, 92%, 93%, 94%, 95%, 96%, 97%, 98%, or 99% identical to SEQ ID NO: 2; (d) an amino acid sequence that is different from the amino acid sequence set forth in SEQ ID NO: 2 by no more than 10, 9, 8, 7, 6, 5, 4, 3, 2 or 1 amino acid; and (e) an amino acid sequence that comprises a substitution, a deletion and/or insertion of one, two, three, four, five or more amino acids to the amino acid sequence set forth in SEQ ID NO: 2.

In one aspect, the disclosure relates to a nucleic acid comprising a nucleotide sequence, wherein the nucleotide sequence is one of the following: (a) a sequence that encodes the protein described herein; (b) SEQ ID NO: 3 (c) SEQ ID NO: 4 (d) SEQ ID NO: 5; (e) SEQ ID NO: 6; (f) SEQ ID NO: 7; (g) SEQ ID NO: 8; (h) SEQ ID NO: 9; (i) SEQ ID NO: 10; (j) a sequence that is at least 90% identical to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10; (k) a sequence that is at least 91%, 92%, 93%, 94%, 95%, 96%, 97%, 98%, or 99% identical to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

In one aspect, the disclosure relates to a cell comprising the protein and/or the nucleic acid described herein.

In one aspect, the disclosure relates to an animal comprising the protein and/or the nucleic acid described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1: Comparison of mouse CCR8 locus and human CCR8 locus (not to scale);

FIG. 2: Schematic diagram of humanization of mouse CCR8 gene (not to scale);

FIG. 3: Schematic diagram of CCR8 gene targeting strategy design (not to scale);

FIG. 4: Southern blot results of cells after CCR8 recombination, where WT is a wild-type control;

FIG. 5: Schematic diagram of humanized CCR8 mouse FRT recombination process (not to scale);

FIGS. 6A-6D: Genotype identification results of F1 generation of CCR8 humanized mice, where WT is wild type, H₂O is water control, and PC is positive control;

FIGS. 7A-7D: Flow cytometry detection results of human CCR8 (hCCR8) and murine CCR8 (mCCR8) in CD4+ T cells, where WT is wild-type C57BL/6 mouse, and H/H is CCR8 humanized homozygous mouse;

FIGS. 8A-8D: Flow cytometry detection results of hCCR8 and mCCR8 in Treg cells, where WT is a wild-type C57BL/6 mouse, and H/H is a CCR8 humanized homozygous mouse.

FIG. 9: Comparison of the immune cells in the spleen of C57BL/6 wild-type mice and CCR8 gene humanized homozygous mice (H/H), among which the immune cells include B cells (B cells) and T cells (T cells), NK cells, CD4+ cells, CD8+ cells, Granulocytes, DC cells, macrophages, monocytes.

FIG. 10: Comparison of the immune cells in the thymus of C57BL/6 wild-type mice and CCR8 gene humanized homozygous mice (H/H), among which the immune cells include B cells, T cells, NK cells, CD4+ cells, CD8+ cells, Granulocytes, DC cells, macrophages, monocytes.

FIG. 11: Comparison of the immune cells in the blood of C57BL/6 wild-type mice and CCR8 gene humanized homozygous mice (H/H), among which the immune cells include B cells and T cells, NK cells, CD4+ cells, CD8+ cells, Granulocytes, DC cells, macrophages, monocytes;

FIG. 12: Comparison of the T cell subpopulations in the spleen of C57BL/6 wild-type mice and CCR8 humanized homozygous mice, among which the T cell subpopulations include CD4+ cells (CD4+ cells) and CD8+ cells (CD8+ cells), Treg cells (Tregs).

FIG. 13: Comparison of the T cell subpopulations in the thymus of C57BL/6 wild-type mice and CCR8 humanized homozygous mice, among which the T cell subpopulations include CD4+ cells (CD4+ cells) and CD8+ cells (CD8+ cells), Treg cells (Tregs).

FIG. 14: Comparison of the T cell subpopulations in the peripheral blood of C57BL/6 wild-type mice and CCR8 humanized homozygous mice, among which the T cell subpopulations include CD4+ cells (CD4+ cells) and CD8+ cells (CD8+ cells), Treg cells (Tregs).

FIG. 15: Mouse colon cancer cell MC38 was implanted into CCR8 humanized mice, and anti-human CCR8 antibody was used for anti-tumor efficacy test (10 mg/kg). The figure shows the body weight of experimental animals in groups G1 and G2.

FIG. 16: The mouse colon cancer cell MC38 was implanted into CCR8 humanized mice, and the anti-human CCR8 antibody was used for anti-tumor efficacy test (10 mg/kg). The figure shows the results of the body weight change of the experimental animals in the G1 and G2 groups.

FIG. 17: Mouse colon cancer cell MC38 was implanted into CCR8 humanized mice, and anti-human CCR8 antibody was used for anti-tumor efficacy test (10 mg/kg). The figure shows the results of tumor volume in experimental animals in groups G1 and G2.

FIGS. 18A-18D are schematic diagrams of the percentages of lymphocyte subpopulations infiltrated in the tumor, where FIG. 18A shows the percentages of mCD3, FIG. 18B shows the percentages of mCD4, FIG. 18C shows the percentages of mCD8, and FIG. 18D shows the percentages of Tregs.

FIGS. 19A-19D are schematic diagrams of the percentages of lymphocyte subpopulations in the peripheral blood, where FIG. 19A shows the percentages of mCD3, FIG. 19B shows the percentages of mCD4, FIG. 19C shows the percentages of mCD8, and FIG. 19D shows the percentages of Tregs.

FIGS. 20A-20C: The leukocyte subpopulation analysis. FIG. 20A shows leukocyte subpopulation in the tumor. FIG. 20B shows leukocyte subpopulation in the spleen cells. FIG. 20C shows leukocyte subpopulation in peripheral blood.

FIGS. 21A-21B: The percentages of CCR8 (CD198) in leukocyte subpopulations in tumors. FIG. 21A shows the percentages of human CCR8. FIG. 21B shows the percentages of mouse CCR8.

FIGS. 22A-22B: The percentages of CCR8 (CD198) in leukocyte subpopulations in spleen cells. FIG. 22A shows the percentages of human CCR8. FIG. 22B shows the percentages of mouse CCR8.

FIGS. 23A-23B: The percentages of CCR8 (CD198) in leukocyte subpopulations in peripheral blood. FIG. 23A shows the percentages of human CCR8. FIG. 23B shows the percentages of mouse CCR8.

FIG. 24 shows the alignment between mouse CCR8 amino acid sequence (NP_031746.1; SEQ ID NO: 1) and human CCR8 amino acid sequence (NP_005192.1; SEQ ID NO: 2).

### DETAILED DESCRIPTION

This disclosure relates to transgenic non-human animal with human or chimeric (e.g., humanized) CCR8 (Chemokine (C—C motif) receptor 8; also known as CD198), and methods of use thereof.

The immune system can differentiate between normal cells in the body and those it sees as “foreign,” which allows the immune system to attack the foreign cells while leaving the normal cells alone. This mechanism sometimes involves regulators of the immune system, e.g., immune checkpoints. These immune regulators can either turn up a signal (co-stimulatory molecules) or inhibit a signal. Because many of these regulators are initiated by ligand-receptor interactions, they can be readily blocked by antibodies against the ligands and/or their receptors.

Experimental animal models are an indispensable research tool for studying the effects of these antibodies (e.g., anti-CCR8 antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullisetal U.S. Pat. No. 4,683,195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N. Y., 1986); each of which is incorporated herein by reference in its entirety.

### CCR8

CCR8 is a CC chemokine receptor, which belongs to G protein-coupled receptors. This gene is mainly expressed in immune cells of lymphoid organs, specifically in Treg cells, TH2 cells, monocytes and NK cells. Human CCR8 has four known ligands: CCL1, CCL8, CCL16, and CCL18 (Barsheshet, Yiftah et al. “CCR8+FOXp3+Treg cells as master drivers of immune regulation.” Proceedings of the National Academy of Sciences 114 (2017): 6086-6091).

CCR8 is mainly expressed in Treg cells and plays an important role in the immunosuppressive function mediated by Treg cells. Studies have shown that CCR8 in tumor-resident Treg cells in breast cancer, colon cancer, lung cancer and other patient tissues is significantly higher than CCR8 in normal tissue Treg cells and CCR8 expression can be ignored in peripheral blood Treg cells. Importantly, no expression of CCR8 was detected in effector immune cells in tumors, including αβT cells, NK cells, γδT cells, and myeloid cells, except that the expression in NKT cells was reduced by 50%. At the same time, the amount of expression of CCR8 in tumor-resident Treg cells is also closely related to clinical prognosis. The expression amount is similar among subtypes of breast cancer. Patients with high expression of CCR8 and Foxp3 at the same time have a significantly reduced disease free and overall survival rate, which indicates that antibodies targeting the CCR8 protein can effectively and selectively deplete tumor-resident Treg cells, thereby making it easier for immune cells to attack tumor cells and achieve the effect of treating tumors. In addition, CCR8 is also selectively expressed in TH2 cells, monocytes and NK cells, and participates in various inflammatory diseases mainly by inducing the migration of inflammatory cells.

In view of the fact that CCR8 may play an important role in the treatment of tumor diseases, in order to further study the relevant biological characteristics, improve the effectiveness of pre-clinical efficacy trials, increase the success rate of research and development, make pre-clinical trials more effective and minimize research and development failures, there is an urgent need in this field to develop non-human animal models involving the CCR8 signaling pathway.

A detailed description of CCR8, and the use of anti-CCR8 antibodies to treat cancers are described, e.g., in Karin N. Chemokines and cancer: new immune checkpoints for cancer therapy. Curr Opin Immunol. 2018 April; 51:140-145; and Villarreal D O, et al. Targeting CCR8 Induces Protective Antitumor Immunity and Enhances Vaccine-Induced Responses in Colon Cancer. Cancer Res. 2018 Sep. 15; 78(18):5340-5348; each of which is incorporated by reference in its entirety.

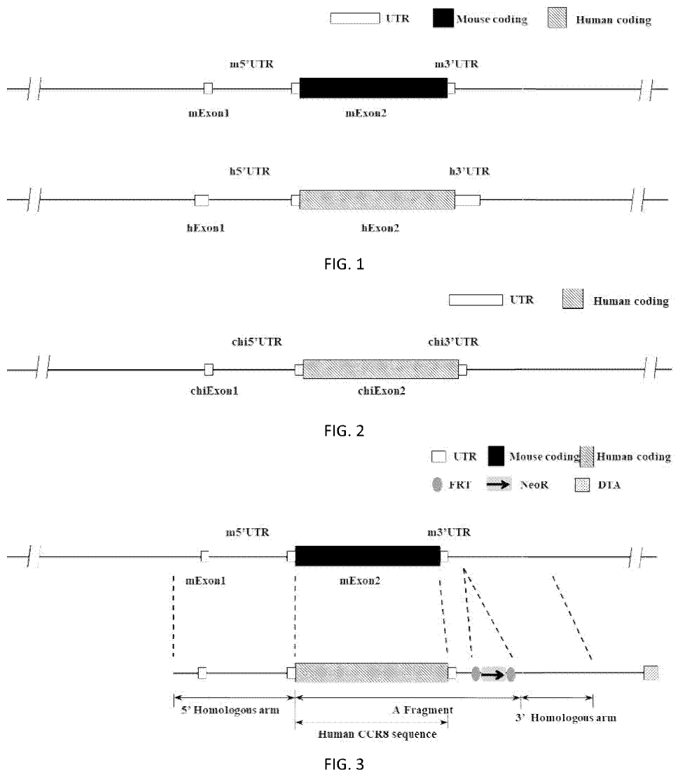

In human genomes, CCR8 gene (Gene ID: 1237) locus has two exons, exon 1 and exon 2 (FIG. 1). The CCR8 protein also has extracellular regions, transmembrane regions, and cytoplasmic regions. The nucleotide sequence for human CCR8 mRNA is NM_005201.4 (SEQ ID NO: 28), and the amino acid sequence for human CCR8 is NP_005192.1 (SEQ ID NO: 2). The location for each exon and each region in human CCR8 nucleotide sequence and amino acid sequence are listed below:

TABLE 1

| | NM_005201.4 | NP_005192.1 |
|---|---|---|
| Human CCR8 | 1484 bp | 355 aa |
| (approximate location) | SEQ ID NO: 28 | SEQ ID NO: 2 |
| Exon 1 | 1-121 | 0 |
| Exon 2 | 122-1484 | 1-355 |
| Signal peptide | — | — |
| Extracellular | 136-240 | 1-35 |
| Extracellular | 415-456 | 94-107 |
| Extracellular | 649-741 | 172-202 |
| Extracellular | 925-975 | 264-280 |
| Transmembrane | 241-324 | 36-63 |
| Transmembrane | 355-414 | 74-93 |
| Transmembrane | 457-522 | 108-129 |
| Transmembrane | 574-648 | 147-171 |
| Transmembrane | 742-801 | 203-222 |
| Transmembrane | 850-924 | 239-263 |
| Transmembrane | 976-1047 | 281-304 |
| Cytoplasmic | 325-354 | 64-73 |
| Cytoplasmic | 523-573 | 130-146 |
| Cytoplasmic | 802-849 | 223-238 |
| Cytoplasmic | 1048-1200 | 305-355 |
| Donor region in Example | 136-1203 | 1-355 |

The human CCR8 gene is located in Chromosome 3 of the human genome, which is located from 39329709 to 39333680 of NC_000003.12 (GRCh38.p13 (GCF_000001405.39)). The 5′-UTR is from 39,329,709 to 39,329,829, and from 39,332,318 to 39,332,331. Exon 1 is from 39,329,709 to 39,329,829, the first intron is from 39,329,830 to 39,332,317, exon 2 is from 39,332,318 to 39,333,680, the 3′-UTR is from 39333400 to 39,333,680, based on transcript NM_005201.4. All relevant information for human CCR8 locus can be found in the NCBI website with Gene ID: 1273, which is incorporated by reference herein in its entirety.

In mice, CCR8 gene locus has two exons, exon 1 and exon 2 (FIG. 1). The mouse CCR8 protein also has extracellular regions, transmembrane regions, and cytoplasmic regions. The nucleotide sequence for mouse CCR8 mRNA is NM_007720.2 (SEQ ID NO: 29), the amino acid sequence for mouse CCR8 is NP_531746.1 (SEQ ID NO: 1). The location for each exon and each region in the mouse CCR8 nucleotide sequence and amino acid sequence is listed below:

TABLE 2

| | NM_007720.2 | NP_031746.1 |
|---|---|---|
| MouseCCR8 | 1156 bp | 353 aa |
| (approximate location) | SEQ ID NO: 29 | SEQ ID NO: 1 |
| Exon 1 | 1-56 | 0 |
| Exon 2 | 57-1156 | 1-353 |
| Signal peptide | — | — |
| Extracellular | 71-169 | 1-33 |
| Extracellular | 344-385 | 92-105 |
| Extracellular | 578-670 | 170-200 |
| Extracellular | 854-904 | 262-278 |
| Transmembrane | 170-253 | 34-61 |
| Transmembrane | 284-343 | 72-91 |
| Transmembrane | 386-451 | 106-127 |
| Transmembrane | 503-577 | 145-169 |
| Transmembrane | 698-730 | 201-220 |
| Transmembrane | 779-853 | 237-261 |
| Transmembrane | 905-976 | 279-302 |
| Cytoplasmic | 254-283 | 62-71 |
| Cytoplasmic | 452-502 | 128-144 |
| Cytoplasmic | 731-778 | 221-236 |
| Cytoplasmic | 977-1129 | 303-353 |
| Replaced region in Example | 71-1132 | 1-353 |

The mouse CCR8 gene (Gene ID: 12776) is located in Chromosome 9 of the mouse genome, which is located from 120092133 to 120094906 of NC_000075.6 (GRCm38.p6 (GCF_000001635.26)). The 5′-UTR is from 120,092,114 to 120,092,188, and from 120,093,807 to 120,093,820, exon 1 is from 120,092,114 to 120,092,188, the first intron is from 120,092,189 to 120,093,806, exon 2 is from 120,093,807 to 120,094,906, the 3′-UTR is from 120094883 to 120,094,906, based on transcript NM_007720.2. All relevant information for mouse CCR8 locus can be found in the NCBI website with Gene ID: 12776, which is incorporated by reference herein in its entirety.

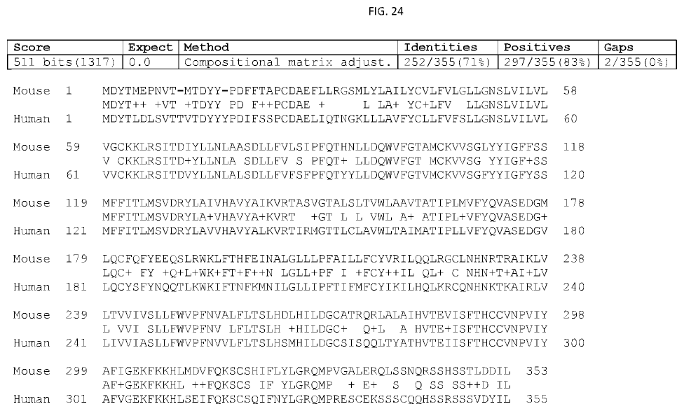

FIG. 24 shows the alignment between mouse CCR8 amino acid sequence (NP_031746.1; SEQ ID NO:1) and human CCR8 amino acid sequence (NP_005192.1; SEQ ID NO:2). Thus, the corresponding amino acid residue or region between human and mouse CCR8 can be found in FIG. 24.

CCR8 genes, proteins, and locus of the other species are also known in the art. For example, the gene ID for CCR8 in Rattus norvegicus is 301066, the gene ID for CCR8 in Macaca mulatta (Rhesus monkey) is 693806, the gene ID for CCR8 in Canis lupus familiaris (dog) is 485600, and the gene ID for CCR8 in Bos taurus (cattle) is 407772. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) CCR8 nucleotide sequence and/or amino acid sequences. In some embodiments, the entire sequence of mouse exon 1, exon 2, extracellular regions, transmembrane regions, and/or cytoplasmic regions are replaced by the corresponding human sequence. In some embodiments, a “region” or “portion” of mouse exon 1, exon 2, extracellular regions, transmembrane regions, and/or cytoplasmic regions are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, one of the extracellular regions (e.g., the 1^{st}, 2^{nd}, 3^{rd} or 4^{th} extracellular region from the N-terminal), one of the transmembrane regions (e.g., the 1^{st}, 2^{nd}, 3^{rd}, 4^{th}, 5^{th}, 6^{th}, or 7^{th} extracellular region from the N-terminal), or one of the cytoplasmic regions (e.g., the 1^{st}, 2^{nd}, 3^{rd}, or 4^{th} cytoplasmic region from the N-terminal). In some embodiments, a region, a portion, or the entire sequence of mouse exon 1 and/or exon 2 (e.g., exon 2) are replaced by the human exon 1 and/or exon 2 (e.g., exon 2) sequence. In some embodiments, a region, a portion, or the entire sequence of one or more of the mouse extracellular regions, one or more of the mouse transmembrane regions, one or more of the mouse cytoplasmic regions are replaced by the corresponding regions in human CCR8.

In some embodiments, the present disclosure also provides a chimeric (e.g., humanized) CCR8 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CCR8 amino acid sequence (e.g., SEQ ID NO: 1), or a portion thereof (e.g., exon 2); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CCR8 amino acid sequence (e.g., SEQ ID NO: 2), or a portion thereof (e.g., exon 2).

In some embodiments, the nucleic acids as described herein are operably linked to a promotor or regulatory element, e.g., an endogenous mouse CCR8 promotor, an inducible promoter, an enhancer, and/or mouse or human regulatory elements.

In some embodiments, the nucleic acids as described herein are operably linked to a Woodchuck Hepatitis Virus (WHP) Posttranscriptional Regulatory Element (WPRE) and/or a polyA (polyadenylation) signal sequence. The WPRE element is a DNA sequence that, when transcribed, creates a tertiary structure enhancing expression. The sequence can be used to increase expression of genes delivered by viral vectors. WPRE is a tripartite regulatory element with gamma, alpha, and beta components.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse CCR8 nucleotide sequence (e.g., exon 1, exon 2, or NM_007720.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse CCR8 nucleotide sequence (e.g., exon 1, exon 2, or NM_007720.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human CCR8 nucleotide sequence (e.g., exon 1, exon 2, or NM_005201.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human CCR8 nucleotide sequence (e.g., exon 1, exon 2, or NM_005201.4).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse CCR8 amino acid sequence (e.g., exon 1, exon 2, or NP_031746.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse CCR8 amino acid sequence (e.g., exon 1, exon 2, or NP_031746.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human CCR8 amino acid sequence (e.g., exon 1, exon 2, or NP_005192.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human CCR8 amino acid sequence (e.g., exon 1, exon 2, or NP_005192.1 (SEQ ID NO: 2)).

The present disclosure also provides a humanized CCR8 mouse amino acid sequence, wherein the amino acid sequence is selected from the group consisting of:
a) an amino acid sequence shown in SEQ ID NO: 2;
b) an amino acid sequence having a homology of at least 90% with or at least 90% identical to the amino acid sequence shown in SEQ ID NO: 2;
c) an amino acid sequence encoded by a nucleic acid sequence, wherein the nucleic acid sequence is able to hybridize to a nucleotide sequence encoding the amino acid shown in SEQ ID NO: 2 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 2;
e) an amino acid sequence that is different from the amino acid sequence shown in SEQ ID NO: 2 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 2.

The present disclosure also relates to a CCR8 nucleic acid (e.g., DNA or RNA) sequence, wherein the nucleic acid sequence can be selected from the group consisting of:
a) a nucleic acid sequence as shown in SEQ ID NO: 5, or SEQ ID NO: 10, or a nucleic acid sequence encoding a homologous CCR8 amino acid sequence of a humanized mouse;
b) a nucleic acid sequence that is shown in SEQ ID NO: 5, or SEQ ID NO: 10;
c) a nucleic acid sequence that is able to hybridize to the nucleotide sequence as shown in SEQ ID NO: 5, or SEQ ID NO: 10 under a low stringency condition or a strict stringency condition;
d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 5, or SEQ ID NO: 10;
e) a nucleic acid sequence that encodes an amino acid sequence, wherein the amino acid sequence has a homology of at least 90% with or at least 90% identical to the amino acid sequence shown in SEQ ID NO: 2;
f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 2;
g) a nucleic acid sequence that encodes an amino acid sequence, wherein the amino acid sequence is different from the amino acid sequence shown in SEQ ID NO: 2 by no more than 10, 9, 8, 7, 6, 5, 4, 3, 2 or no more than 1 amino acid; and/or
h) a nucleic acid sequence that encodes an amino acid sequence, wherein the amino acid sequence comprises a substitution, a deletion and/or insertion of one or more amino acids to the amino acid sequence shown in SEQ ID NO: 2.

The present disclosure further relates to a CCR8 genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 5, or SEQ ID NO: 10.

The disclosure also provides an amino acid sequence that has a homology of at least 90% with, or at least 90% identical to the sequence shown in SEQ ID NO: 2, and has protein activity. In some embodiments, the homology with the sequence shown in SEQ ID NO: 2 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 2 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 5, or SEQ ID NO: 10, and encodes a polypeptide that has protein activity. In some embodiments, the homology with the sequence shown in SEQ ID NO: 5, or SEQ ID NO: 10 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 5, or SEQ ID NO: 10 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) CCR8 from an endogenous non-human CCR8 locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous CCR8 locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wildtype nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wildtype amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized CCR8 gene or a humanized CCR8 nucleic acid. In some embodiments, at least one or more portions of the gene or the nucleic acid is from the human CCR8 gene, at least one or more portions of the gene or the nucleic acid is from a non-human CCR8 gene. In some embodiments, the gene or the nucleic acid comprises a sequence that encodes a CCR8 protein. The encoded CCR8 protein is functional or has at least one activity of the human CCR8 protein or the non-human CCR8 protein, e.g., binding with human or non-human CCL1, CCL8, CCL16, and/or CCL18, decreasing the level of activation of immune cells (e.g., T cells), reducing apoptosis in regulatory T cells, promoting apoptosis in antigen-specific T-cells in lymph nodes, and/or downregulating the immune response.

In some embodiments, the chimeric protein or the chimeric polypeptide is a humanized CCR8 protein or a humanized CCR8 polypeptide. In some embodiments, at least one or more portions of the amino acid sequence of the protein or the polypeptide is from a human CCR8 protein, and at least one or more portions of the amino acid sequence of the protein or the polypeptide is from a non-human CCR8 protein. The humanized CCR8 protein or the humanized CCR8 polypeptide is functional or has at least one activity of the human CCR8 protein or the non-human CCR8 protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains.

In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, L E A, Sprague Dawley, Fischer, F344, F6, and Dark Agouti.

The animal can have one or more other genetic modifications, and/or other modifications, that are suitable for the particular purpose for which the humanized CCR8 animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γc null mice (Ito, M. et al., NOD/SCID/γc null mouse: an excellent recipient mouse model for engraftment of human cells, Blood 100 (9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human CCR8 locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature CCR8 coding sequence with human mature CCR8 coding sequence or an insertion of human mature CCR8 coding sequence or chimeric CCR8 coding sequence.

Genetically modified non-human animals that comprise a modification of an endogenous non-human CCR8 locus. In some embodiments, the modification can comprise a human nucleic acid sequence encoding at least a portion of a mature CCR8 protein (e.g., at least 10%, 20%, 30%, 40%, 50%, 60%, 70%, 80%, 90%, 95%, 96%, 97%, 98%, or 99% identical to the mature CCR8 protein sequence). Although genetically modified cells are also provided that can comprise the modifications described herein (e.g., ES cells, somatic cells), in many embodiments, the genetically modified non-human animals comprise the modification of the endogenous CCR8 locus in the germline of the animal.

Genetically modified animals can express a human CCR8 and/or a chimeric (e.g., humanized) CCR8 from endogenous mouse loci, wherein the endogenous mouse CCR8 gene has been replaced with a human CCR8 gene and/or a nucleotide sequence that encodes a region of human CCR8 sequence or an amino acid sequence that is at least 10%, 20%, 30%, 40%, 50%, 60%, 70&, 80%, 90%, 95%, 96%, 97%, 98%, or 99% identical to the human CCR8 sequence. In various embodiments, an endogenous non-human CCR8 locus is modified in whole or in part to comprise human nucleic acid sequence encoding at least one protein-coding sequence of a mature CCR8 protein.

In some embodiments, the genetically modified mice express the human CCR8 and/or chimeric CCR8 (e.g., humanized CCR8) from endogenous loci that are under control of mouse promoters and/or mouse regulatory elements. The replacement(s) at the endogenous mouse loci provide non-human animals that express human CCR8 or chimeric CCR8 (e.g., humanized CCR8) in appropriate cell types and in a manner that does not result in the potential pathologies observed in some other transgenic mice known in the art. The human CCR8 or the chimeric CCR8 (e.g., humanized CCR8) expressed in animal can maintain one or more functions of the wildtype mouse or human CCR8 in the animal. For example, human or non-human CCR8 ligands (e.g., CCL1, CCL8, CCL16, and/or CCL18) can bind to the expressed CCR8, downregulate immune response, e.g., downregulate immune response by at least 10%, 20%, 30%, 40%, or 50%. Furthermore, in some embodiments, the animal does not express endogenous CCR8. As used herein, the term “endogenous CCR8” refers to CCR8 protein that is expressed from an endogenous CCR8 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human CCR8 (NP_005192.1) (SEQ ID NO: 2). In some embodiments, the genome comprises a sequence encoding an amino acid sequence that is at least 70%, 75%, 80%, 85%, 90%, 95%, 99%, or 100% identical to SEQ ID NO: 2.

The genome of the genetically modified animal can comprise a replacement at an endogenous CCR8 gene locus of a sequence encoding a region of endogenous CCR8 with a sequence encoding a corresponding region of human CCR8. In some embodiments, the sequence that is replaced is any sequence within the endogenous CCR8 gene locus, e.g., exon 1, exon 2, 5′-UTR, 3′-UTR, the first intron, sequences encoding the 1^{st}, 2^{nd}, 3^{rd}, or 4^{th} extracellular region from the N-terminal, sequences encoding the 1^{st}, 2^{nd}, 3^{rd}, 4^{th}, 5^{th}, 6^{th}, or 7^{th} extracellular region from the N-terminal, and/or sequences encoding the 1^{st}, 2^{nd}, 3^{rd} or 4^{th} cytoplasmic region from the N-terminal etc. In some embodiments, the sequence that is replaced is within the regulatory region of the endogenous CCR8 gene. In some embodiments, the sequence that is replaced is exon 2 or part thereof, of an endogenous mouse CCR8 gene locus.

In some embodiments, a sequence that encodes an amino acid sequence (e.g., human CCR8 or chimeric CCR8) is inserted immediately after 5′-UTR or at the start codon (e.g., within 10, 20, 30, 40, 50, 60, 70, 80, 90, or 100 nucleic acids). The start codon is the first codon of a messenger RNA (mRNA) transcript translated by a ribosome. The start codon always codes for methionine in eukaryotes and a modified Met (fMet) in prokaryotes. The most common start codon is ATG (or AUG in mRNA).

In some embodiments, the inserted sequence further comprises a stop codon (e.g., TAG, TAA, TGA). The stop codon (or termination codon) is a nucleotide triplet within messenger RNA that signals a termination of translation into proteins. Thus, the endogenous sequence after the stop codon will not be translated into proteins. In some embodiments, at least one exons of (e.g., exon 1 and/or exon 2) of the endogenous CCR8 gene are not translated into proteins.

The genetically modified animal can have one or more cells expressing a human or chimeric CCR8 (e.g., humanized CCR8) having e.g., humanized extracellular regions and/or humanized cytoplasmic regions, wherein one or more of the extracellular regions comprises a sequence that is at least 50%, 60%, 70%, 80%, 90%, 95%, 99% identical to the corresponding extracellular region of human CCR8. In some embodiments, one or more of the extracellular regions of the humanized CCR8 has a sequence that has at least 10, 20, 30, 40, 50, 60, 70, 80, 90, 100, 110, 120, 130, 140, 150, 160, 170, or 180 amino acids (e.g., contiguously or non-contiguously) that are identical to the corresponding extracellular region of human CCR8. Because human CCR8 and non-human CCR8 (e.g., mouse CCR8) sequences, in many cases, are different, antibodies that bind to human CCR8 will not necessarily have the same binding affinity with non-human CCR8 or have the same effects to non-human CCR8. Therefore, the genetically modified animal having human or a humanized extracellular regions can be used to better evaluate the effects of anti-human CCR8 antibodies in an animal model. In some embodiments, the genome of the genetically modified animal comprises a sequence encoding an amino acid sequence that corresponds to part or the entire sequence of exon 1 and/or exon 2 of human CCR8, part or the entire sequence of extracellular regions of human CCR8, or part or the entire sequence of SEQ ID NO: 2.

In some embodiments, the non-human animal can have, at an endogenous CCR8 gene locus, a nucleotide sequence encoding a chimeric human/non-human CCR8 polypeptide, wherein a human portion of the chimeric human/non-human CCR8 polypeptide comprises a portion of a human CCR8 extracellular region, and wherein the animal expresses a functional CCR8 on a surface of a cell of the animal. The human portion of the chimeric human/non-human CCR8 polypeptide can comprise a portion of exon 1 and/or exon 2 of human CCR8, or one or more human CCR8 transmembrane regions. In some embodiments, the human portion of the chimeric human/non-human CCR8 polypeptide can comprise a sequence that is at least 80%, 85%, 90%, 95%, or 99% identical to SEQ ID NO: 2.

In some embodiments, the non-human animal genome also includes other genetic modifications. In some embodiments, the other genes include one of human PD-1, PD-L1, CTLA4, LAG3, IL4, IL6, or CCR4 genes, or a combination of two or more.

In some embodiments, the nucleotide sequence of the humanized CCR8 gene includes one of the following groups:
(1) all or part of the nucleotide sequence shown in SEQ ID NO: 5;
(2) at least 60%, 65%, 70%, 75%, 80%, 85%, 90%, 91%, 92%, 93% identity with the nucleotide sequence shown in SEQ ID NO: 5, 94%, 95%, 96%, 97%, 98% or at least 99%;
(3) no more than 10, 9, 8, 7, 6, 5, 4, 3, 2 or no more than 1 nucleotide difference from the nucleotide sequence shown in SEQ ID NO: 5; or
(4) the nucleotide sequence shown in the nucleotide sequence shown in SEQ ID NO: 5, including substitution, deletion and/or insertion of one or more nucleotides.

In some embodiments, the humanized CCR8 gene further comprises an auxiliary sequence, which is connected after the human CCR8 gene. Further preferably, the auxiliary sequence is selected from a stop codon, a flip sequence or a knockout sequence. More preferably, the auxiliary sequence is 3′UTR and/or polyA of a non-human animal.

In some embodiments, the non-human animal can have transcribed mRNA sequence including one of the following groups:
(1) all or part of the nucleotide sequence shown in SEQ ID NO: 10;
(2) at least 60%, 65%, 70%, 75%, 80%, 85%, 90%, 91%, 92%, 93% identity with the nucleotide sequence shown in SEQ ID NO: 10, 94%, 95%, 96%, 97%, 98% or at least 99%;
(3) no more than 10, 9, 8, 7, 6, 5, 4, 3, 2 or no more than 1 nucleotide difference from the nucleotide sequence shown in SEQ ID NO: 10; or
(4) the nucleotide sequence shown in the nucleotide sequence shown in SEQ ID NO: 10, including substitution, deletion and/or insertion of one or more nucleotides.

In some embodiments, the humanized CCR8 gene also includes a specific inducer or repressor. Further preferably, the specific inducer or repressor can be a conventional inducing or repressing substance.

In a specific embodiment of the present invention, the specific inducer is selected from the tetracycline system (Tet-Off System/Tet-On System) or the tamoxifen system (Tamoxifen System).

In some embodiments, the non-human portion of the chimeric human/non-human CCR8 polypeptide comprises a transmembrane region and/or a cytoplasmic region of an endogenous non-human CCR8 polypeptide. There may be several advantages that are associated with the transmembrane and/or cytoplasmic regions of an endogenous non-human CCR8 polypeptide. For example, once a CCR8 ligand (e.g., CCL1) or an anti-CCR8 antibody binds to CCR8, they can properly transmit extracellular signals into the cells and initiate the downstream pathway.

Furthermore, the genetically modified animal can be heterozygous with respect to the replacement or insertion at the endogenous CCR8 locus, or homozygous with respect to the replacement or insertion at the endogenous CCR8 locus.

In some embodiments, the genetically modified animal (e.g., a rodent) comprises a humanization of an endogenous CCR8 gene, wherein the humanization comprises a replacement at the endogenous rodent CCR8 locus of a nucleic acid comprising an exon, or a part thereof, of a CCR8 gene with a nucleic acid sequence comprising at least one exon, or a part thereof, of a human CCR8 gene to form a modified CCR8 gene.

In some embodiments, the genetically modified animal (e.g., a rodent) comprises an insertion at the endogenous rodent CCR8 locus of a nucleic acid sequence comprising at least one exon of a human CCR8 gene to form a modified CCR8 gene In some embodiments, the expression of the modified CCR8 gene is under control of regulatory elements at the endogenous CCR8 locus (e.g., 5′-UTR or 3′-UTR).

In some embodiments, the humanized CCR8 locus lacks a human CCR8 5′-UTR. In some embodiment, the humanized CCR8 locus comprises a rodent (e.g., mouse) 5′-UTR. In some embodiments, the humanization comprises a human 3′-UTR. In appropriate cases, it may be reasonable to presume that the mouse and human CCR8 genes appear to be similarly regulated based on the similarity of their 5′-flanking sequence. As shown in the present disclosure, humanized CCR8 mice that comprise a replacement at an endogenous mouse CCR8 locus, which retain mouse regulatory elements but comprise a humanization of CCR8 encoding sequence, do not exhibit obvious pathologies. Both genetically modified mice that are heterozygous or homozygous for humanized CCR8 are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized CCR8 gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized CCR8 in the genome of the animal.

In some embodiments, the non-human mammal comprises the genetic construct as described herein (e.g., gene construct as shown in FIG. 3). In some embodiments, a non-human mammal expressing human or humanized CCR8 is provided. In some embodiments, the tissue-specific expression of human or humanized CCR8 protein is provided.

In some embodiments, the expression of human or humanized CCR8 in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human CCR8 protein or chimeric CCR8 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNA dot) analysis. Immunohistochemical staining, flow cytometry, Western blot analysis can also be used to assess the presence of human or humanized CCR8 protein.

### Vectors

The present disclosure relates to a targeting vector, comprising: a) a DNA fragment homologous to the 5′ end of a region to be altered (5′ arm), which is selected from the CCR8 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the CCR8 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000075.6; c) the DNA fragment homologous to the 3′ end of the region to be altered (3′ arm) is selected from the nucleotide sequences that have at least 90% homology to the NCBI accession number NC_000075.6.

In some embodiments, a) the DNA fragment homologous to the 5′ end of a region to be altered (5′ arm) is selected from the nucleotides from the position 120088585 to the position 120093820 of the NCBI accession number NC_000075.6; c) the DNA fragment homologous to the 3′ end of the region to be altered (3′ arm) is selected from the nucleotides from the position 120095301 to the position 120099151 of the NCBI accession number NC_000075.6.

In some embodiments, the length of the selected genomic nucleotide sequence in the targeting vector can be more than about 0.6 kb, about 0.8 kb, about 1 kb, or about 2 kb.

In some embodiments, the region to be altered is exon 1 and/or exon 2 of CCR8 gene (e.g., exon 2 of mouse CCR8 gene).

The targeting vector can further include a selected gene marker.

In some embodiments, the sequence of the 5′ arm is shown in SEQ ID NO: 3; and the sequence of the 3′ arm is shown in SEQ ID NO: 4.

In some embodiments, the sequence is derived from human. For example, the target region in the targeting vector is a part or entirety of the nucleotide sequence of a human CCR8 or a chimeric CCR8. In some embodiments, the nucleotide sequence of the humanized CCR8 encodes the entire or the part of human CCR8 protein with the NCBI accession number NP_005192.1 (SEQ ID NO: 2).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

Methods of making genetically modified animals Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous CCR8 gene locus, a sequence encoding a region of an endogenous CCR8 with a sequence encoding a corresponding region of human CCR8, a sequencing encoding human CCR8, or a sequencing encoding chimeric CCR8.

In some embodiments, the disclosure provides inserting in at least one cell of the animal, at an endogenous CCR8 gene locus, a sequence encoding a human CCR8 or a chimeric CCR8.

In some embodiments, the genetic modification occurs in a germ cell, a somatic cell, a blastocyst, or a fibroblast, etc. The nucleus of a somatic cell or the fibroblast can be inserted into an enucleated oocyte.

FIG. 3 shows a humanization strategy for a mouse CCR8 locus. In FIG. 3, the targeting strategy involves a vector comprising the 5′ end homologous arm, human CCR8 gene fragment or chimeric CCR8 gene fragment, 3′ homologous arm. The process can involve replacing endogenous CCR8 sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous CCR8 sequence with human CCR8 sequence.

Thus, in some embodiments, the methods for making a genetically modified, humanized animal, can include the step of replacing at an endogenous CCR8 locus (or site), a nucleic acid encoding a sequence encoding a region of endogenous CCR8 with a sequence encoding a human CCR8 or a chimeric CCR8. The sequence can include a region (e.g., a part or the entire region) of exon 1, exon 2 of a human CCR8 gene. In some embodiments, the sequence includes a region of exon 1, exon 2 of a human CCR8 gene (e.g., SEQ ID NO: 2). In some embodiments, the region is located within the extracellular regions of CCR8. In some embodiments, the endogenous CCR8 locus is exon 1 and/or exon 2 of mouse CCR8 (e.g., exon 2).

In some embodiments, the methods of modifying a CCR8 locus of a mouse to express a chimeric human/mouse CCR8 peptide can include the steps of replacing at the endogenous mouse CCR8 locus a nucleotide sequence encoding a mouse CCR8 with a nucleotide sequence encoding a human CCR8, thereby generating a sequence encoding a chimeric human/mouse CCR8.

The present disclosure further provides a method for establishing a CCR8 gene humanized animal model, involving the following steps:
(a) providing the cell (e.g. a fertilized egg cell) based on the methods described herein; (b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized CCR8 protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized CCR8, which are useful for testing agents that can decrease or block the interaction between CCR8 and CCR8 ligands (e.g., CCL1, CCL8, CCL16, and/or CCL18) or the interaction between CCR8 and anti-human CCR8 antibodies, testing whether an agent can increase or decrease the immune response, and/or determining whether an agent is an CCR8 agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In some embodiments, the genetically modified animals can be used for determining effectiveness of a CCR8 inhibitor for the treatment of cancer. The methods involve administering the CCR8 inhibitor (e.g., anti-human CCR8 antibody or anti-human CCL1 antibody) to the animal as described herein, wherein the animal has a tumor; and determining the inhibitory effects of the CCR8 inhibitor to the tumor. In some embodiments, the CCR8 inhibitor is an anti-human CCR8 antibody or anti-human CCL1 antibody.

The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the tumor comprises one or more cancer cells (e.g., human or mouse cancer cells) that are injected into the animal. In some embodiments, the anti-CCR8 antibody, anti-CCL1 antibody, anti-CCL8 antibody, anti-CCL16 antibody or anti-CCL18 antibody prevents CCR8 ligands from binding to CCR8. In some embodiments, the anti-CCR8 antibody, anti-CCL1 antibody, anti-CCL8 antibody, anti-CCL16 antibody or anti-CCL18 antibody does not prevent the ligands from binding to CCR8.

In some embodiments, the genetically modified animals can be used for determining whether an anti-CCR8 antibody is a CCR8 agonist or antagonist. In some embodiments, the methods as described herein are also designed to determine the effects of the agent (e.g., anti-CCR8 antibodies) on CCR8, e.g., whether the agent can stimulate immune cells or inhibit immune cells (e.g., T cells), whether the agent can increase or decrease the production of cytokines, whether the agent can activate or deactivate immune cells (e.g., T cells, macrophages, B cells, or DC), whether the agent can upregulate the immune response or downregulate immune response, and/or whether the agent can induce complement mediated cytotoxicity (CMC) or antibody dependent cellular cytoxicity (ADCC). In some embodiments, the genetically modified animals can be used for determining the effective dosage of a therapeutic agent for treating a disease in the subject, e.g., cancer, or autoimmune diseases.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the anti-CCR8 antibody or the anti-CCL1 antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the anti-CCR8 antibody is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the anti-CCR8 antibody is designed for treating hepatocellular, ovarian, colon, or cervical carcinomas. In some embodiments, the anti-CCR8 antibody is designed for treating advanced breast cancer, advanced ovarian cancer, and/or advanced refractory solid tumor. In some embodiments, the anti-CCR8 antibody is designed for treating metastatic solid tumors, NSCLC, melanoma, non-Hodgkin lymphoma, colorectal cancer, and multiple myeloma. In some embodiments, the anti-CCR8 antibody is designed for treating melanoma, pancreatic carcinoma, mesothelioma, hematological malignancies (e.g., Non-Hodgkin's lymphoma, lymphoma, chronic lymphocytic leukemia), or solid tumors (e.g., advanced solid tumors). In some embodiments, the anti-CCR8 antibody is designed for treating carcinomas (e.g., nasopharynx carcinoma, bladder carcinoma, cervix carcinoma, kidney carcinoma or ovary carcinoma).

In some embodiments, the anti-CCR8 antibody is designed for treating various autoimmune diseases. The “autoimmune diseases” include but are not limited to allergies, asthma, myocarditis, nephritis, hepatitis, systemic lupus erythematosus, rheumatoid arthritis, scleroderma, hyperthyroidism, primary thrombocytopenia Purpura, autoimmune hemolytic anemia, ulcerative colitis, autoimmune liver disease, diabetes, pain or neurological disorders, etc. The methods as described herein can be used to determine the effectiveness of an anti-CCR8 antibody in inhibiting immune response.

In some embodiments, the anti-CCR8 antibody is designed for treating various inflammation. The “inflammation” includes e.g., acute inflammation and also chronic inflammation. Specifically, including but not limited to degenerative inflammation, exudative inflammation (serous inflammation, fibrinitis, suppurative inflammation, hemorrhagic inflammation, necrotitis, catarrhal inflammation), proliferative inflammation, specific inflammation (Tuberculosis, syphilis, leprosy, lymphogranuloma, etc.).

The present disclosure also provides methods of determining toxicity of an antibody (e.g., anti-CCR8 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the CCR8 gene function, human CCR8 antibodies, drugs for human CCR8 targeting sites, the drugs or efficacies for human CCR8 targeting sites, the drugs for immune-related diseases and antitumor drugs.

Genetically Modified Animal Model with Two or More Human or Chimeric Genes

The present disclosure further relates to methods for generating genetically modified animal model with two or more human or chimeric genes. The animal can comprise a human or chimeric CCR8 gene and a sequence encoding an additional human or chimeric protein.

In some embodiments, the additional human or chimeric protein can be cytotoxic T-lymphocyte-associated protein 4 (CTLA-4), Lymphocyte Activating 3 (LAG-3), B And T Lymphocyte Associated (BTLA), Programmed cell death protein 1 (PD-1), Programmed death-ligand 1 (PD-L1), CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), or TNF Receptor Superfamily Member 4 (TNFRSF4 or OX40).

The methods of generating genetically modified animal model with two or more human or chimeric genes (e.g., humanized genes) can include the following steps:
(a) using the methods of introducing human CCR8 gene or chimeric CCR8 gene as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric CTLA-4, LAG-3, BTLA, PD-1, PD-L1, CD27, CD28, CD40, CD47, CD137, CD154, TIGIT, TIM-3, GITR, SIRPa, or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the CCR8 humanization is directly performed on a genetically modified animal having a human or chimeric CTLA-4, BTLA, PD-1, PD-L1, CD27, CD28, CD40, CD47, CD137, CD154, TIGIT, TIM-3, GITR, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-CCR8 antibody and an additional therapeutic agent for the treatment of cancer. The methods include administering the anti-CCR8 antibody and the additional therapeutic agent to the animal, wherein the animal has a tumor; and determining the inhibitory effects of the combined treatment to the tumor. In some embodiments, the additional therapeutic agent is an antibody that specifically binds to CTLA-4, BTLA, PD-1, PD-L1, CD27, CD28, CD40, CD47, CD137, CD154, TIGIT, TIM-3, GITR, or OX40. In some embodiments, the additional therapeutic agent is an anti-PD-1 antibody (e.g., nivolumab, pembrolizumab).

In some embodiments, the combination treatment is designed for treating various cancer as described herein, e.g., melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, the combination treatment is designed for treating metastatic solid tumors, NSCLC, melanoma, B-cell non-Hodgkin lymphoma, colorectal cancer, and multiple myeloma. In some embodiments, the combination treatment is designed for treating melanoma, carcinomas (e.g., pancreatic carcinoma), mesothelioma, hematological malignancies (e.g., Non-Hodgkin's lymphoma, lymphoma, chronic lymphocytic leukemia), or solid tumors (e.g., advanced solid tumors).

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

EcoNI, ScaI, and DraIII enzymes were purchased from NEB, and the catalog numbers are R0521, R3122, R3510, respectively;

Lipopolysaccharide from Escherichia coli O111:B4 was purchased from Sigma, with the catalog number: L2630;

Attune Nxt Acoustic Focusing Cytometer purchased from Thermo Fisher, Model #Attune Nxt;

PrimeScript 1st Strand cDNA Synthesis Kit was purchased from TAKARA, Model #6110A;

Heraeus™ Fresco™ 21 microcentrifuge was purchased from Thermo Fisher, Model #Fresco 21;

PerCP anti-mouse CD45 antibody was purchased from Biolegend, with the catalog #03130;

Brilliant Violet 711™ anti-mouse TCRP chain antibody was purchased from Biolegend, with the catalog #109243;

Brilliant Violet 510™ anti-mouse CD4 antibody was purchased from Biolegend, with the catalog #100559;

FITC anti-mouse CD8a antibody was purchased from Biolegend, with the catalog #100706;

Brilliant Violet 421™ anti-mouse NK1.1 antibody was purchased from Biolegend, with the catalog #108732;

FITC anti-mouse F4/80 antibody was purchased from Biolegend, with the catalog #123108;

FITC anti-human CD3 antibody was purchased from Biolegend, with the catalog #300306;

APC anti-mouse CD198 (CCR8) antibody was purchased from Biolegend, with the catalog #150309;

PE anti-human CD198 (CCR8) antibody was purchased from Biolegend, with the catalog #360603;

PerCP anti-mouse/human CD11b antibody was purchased from Biolegend, with the catalog #101230.

### Example 1. CCR8 Humanized Mice

In this example, a non-human animal (such as a mouse) is modified so that the non-human animal contains a nucleotide sequence encoding a humanized CCR8 protein to obtain a genetically modified non-human animal that can express the humanized CCR8 protein. Mouse CCR8 gene (NCBI Gene ID: 12776, Primary source: MGI: 1201402, UniProt ID: P56484, located at 120092133 to 12094906 of chromosome 9 NC_000075.6, based on the transcript NM_007720.2 and its encoded protein NP_031746.1 (SEQ ID NO:1) and human CCR8 gene (NCBI Gene ID: 1237, Primary source: HGNC: 1609, UniProt ID: P51685, located at positions 39329709 to 39333680 on chromosome 3 NC_000003.12, based on the transcript NM 005201.4 and its coding protein NP_005192.1 (SEQ ID NO: 2)) were used. The comparison of the mouse CCR8 locus and the human CCR8 locus are shown in FIG. 1.

In order to make a humanized CCR8 mouse, a part of the nucleotide sequence encoding the human CCR8 protein can be introduced into the mouse endogenous CCR8 locus, so that the mouse expresses the human or humanized CCR8 protein. Specifically, a 1062 bp nucleotide sequence of exon 2 of the mouse CCR8 gene can be replaced with the 1068 bp nucleotide sequence of the corresponding part of exon 2 of the human CCR8 gene by gene editing to obtain the humanized CCR8 gene (schematic diagram shown in FIG. 2).

In the schematic diagram of the targeting strategy shown in FIG. 3, the targeting vector contains the homologous arm sequences upstream and downstream of the mouse CCR8 gene, and the A fragment containing the human CCR8 gene sequence. Among them, the upstream homology arm sequence (5′ homology arm, SEQ ID NO: 3) is the same as nucleotide 120088585-120093820 of NCBI accession number NC_000075.6, and the downstream homology arm sequence (3′ homology Arm, SEQ ID NO: 4) is the same as nucleotide 120095301-120099151 of NCBI accession number NC_000075.6. The A segment contains a partial sequence of exon 2 of the human CCR8 gene (SEQ ID NO: 5). This DNA sequence is the same as the nucleotide 39332332-39333399 of NCBI accession number NC_000003.12. The connection between the human CCR8 sequence upstream of the A fragment and the mouse CCR8 gene is designed to be 5′-acctctcacgtgcctgcttgaccaggtcttcctgcctcgATGGATTATACACTTGACCTCAGTGTGACAACA GTGACCGA-3′ (SEQ ID NO: 6), where the last “g” in the sequence “gcctcg” is the last mouse nucleotide, and the first “A” in the sequence “ATGGA” is the first human nucleotide. The connection between the downstream of the human CCR8 sequence and the mouse CCR8 gene is designed as 5′-CTCCTCCCGTTCCTCCAGCGTAGACTACATTTTGTGAggggagtgtgcagggcaggcag actccacctgcattgcccttcc-3′ (SEQ ID NO: 7), where the last “A” in the sequence “TGTGA” is the last human nucleotide, and the first “g” in the sequence “gggga” is the first mouse nucleotide.

The targeting vector also includes the resistance gene used for positive clone screening, namely the neomycin phosphotransferase coding sequence Neo, and two site-specific Frt recombination systems arranged in the same direction are installed on both sides of the resistance gene. The recombination site constitutes a Neo cassette. The connection between the 5′ end of the Neo cassette and the mouse gene is designed as: 5′-agatggctctgcagctaaaggcacttgttcttactAAGCTTGATATCGAATTCCGAAGTTCCTATTCTCTAG AAAGTATAGGAACTTC-3′ (SEQ ID NO: 8), where the last “t” in the sequence “ttact” is the last mouse nucleotide, and the first “A” in the sequence “AAGCT” is the first nucleotide of the Neo cassette. The connection between the 3′ end of the Neo cassette and the mouse gene is designed as 5′-GAAGTTCCTATTCTCTAGAAAGTATAGGAACTTCATCAGTCA GGTACATAATTAGGTGGATCCACTAGTTCTAGActgagaactggacccaggggctggagagatggctcag t-3′ (SEQ ID NO: 9), where the last “A” in the sequence “CTAGA” is the last nucleotide of the Neo cassette, and the “c” in the sequence “ctgag” is the first mouse nucleotide. In addition, a coding gene with a negative selection marker (coding gene for diphtheria toxin A subunit (DTA)) was added downstream of the 3′ homology arm of the targeting vector. The mRNA sequence of the humanized mouse CCR8 is shown in SEQ ID NO: 10, and the expressed protein sequence is shown in SEQ ID NO: 2.

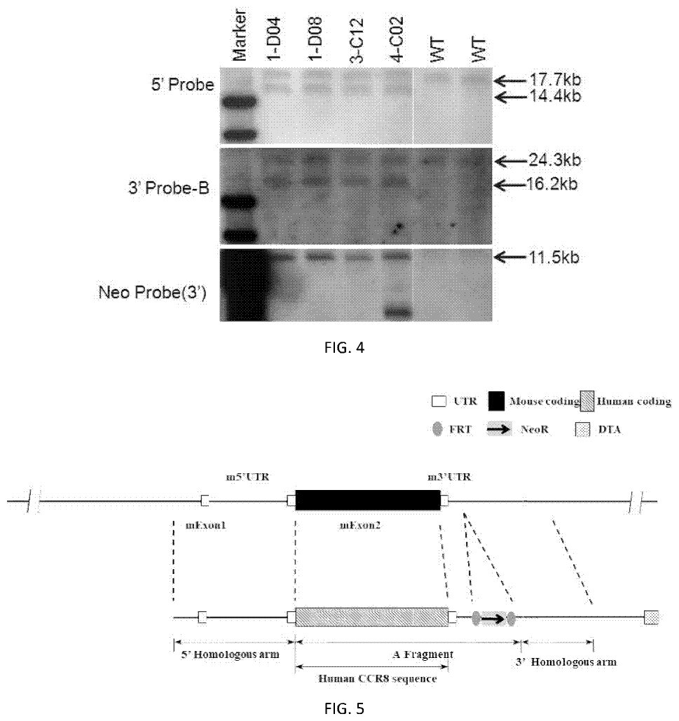

The construction of the targeting vector can be carried out by e.g., restriction enzyme digestion and ligation. The constructed targeting vector was initially verified by restriction digestion, and then sent to a sequencing company for sequencing verification. The targeted vector verified by sequencing was electroporated into embryonic stem cells of C57BL/6 mice, and the obtained cells were screened using positive clone selection marker genes. PCR (PCR amplification primers shown in Table 3) and Southern Blot were used to detect and confirm the integration of exogenous genes, and to screen out the correct positive clones. Specifically, Southern Blot was performed on the positive clones identified by PCR (digesting the cell DNA with SspI or SpeI or EcoNI respectively and using 3 probes for hybridization). The length of the PCR probes and the target fragments are shown in Table 4. The PCR results are shown in FIG. 4. The PCR identified 3 clones (the only exception being 4-C02) as positive. Sequencing confirmed that these 3 clones (i.e., 1-D04, 1-D08 and 3-C12) were positive and did not have random insertion.

TABLE 3
PCR amplification primer sequence

| | | | Fragment |
|---|---|---|---|
| Primer | SEQ ID | Sequence(5′-3′) | size(bp) |
| F1 | SEQ ID NO: 11 | TGCTAGTGGAAGACAGACTT | 6065 |
| | | CCAG | |
| Mut-R | SEQ ID NO: 12 | CAGCAAGGAGCAACTTGCCA | |
| | | TTTG | |
| F2 | SEQ ID NO: 13 | GCTCGACTAGAGCTTGCGGA | 4169 |
| R2 | SEQ ID NO: 14 | CTCACGAGCCATCTGAAACC | |
| | | CTGTT | |

TABLE 4
Specific probes and target fragment length

| | | WT target | Target fragment size |
|---|---|---|---|
| Endonuclease | Probe | fragment size | after recombination |
| SspI | 5′ Probe | 17.7 kb | 14.4 kb |
| SpeI | 3′ Probe-B | 24.3 kb | 16.2 kb |
| EcoNI | Neo Probe (3′) | — | 11.5 kb |

Southern Blot detection includes the following probe primers:

5′Probe:
5′Probe-F (SEQ ID NO: 15):
5′-CTGCTCTCACACCATCCTGACTGAG-3′,
5′Probe-R (SEQ ID NO: 16):
5′-TCTCACTGAACTTAGAGCCATCTGTGA-3′;
3′Probe (3′Probe-B):
3′Probe-B-F (SEQ ID NO: 17):
5′-CTCAGCCAGTCACCCATTTCAAAAAGC-3′,
3′Probe-B-R (SEQ ID NO: 18):
5′-GTCTCCACCGTGTCCTCTACATCCTG-3′;
Neo Probe (Neo Probe (3′)):
Neo Probe (3′)-F (SEQ ID NO: 19):
5′-GGATCGGCCATTGAACAAGAT-3′,
Neo Probe (3′)-R (SEQ ID NO: 20):
5′-CAGAAGAACTCGTCAAGAAGGC-3′.

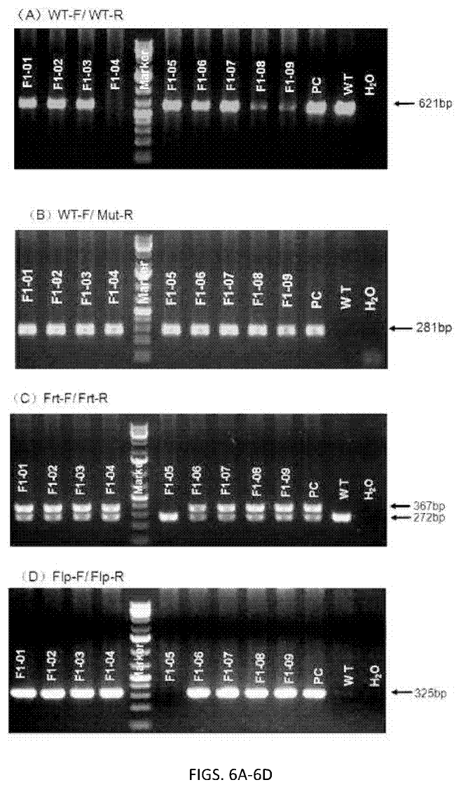

The identified correct positive clone cells (black mouse) were introduced into isolated blastocysts (white mouse) according to techniques known in the art, and the chimeric blastocysts obtained were transferred to a culture medium for short-term culture and then transplanted to the fallopian tubes of recipient female mice (white mice) to produce F0 chimeric mice (black and white). F0 generation chimeric mice and wild-type mice are backcrossed to obtain F1 generation mice, and then F1 generation heterozygous mice were mated with each other to obtain F2 generation homozygous mice. The mouse was further mated with Flp tool mice to remove the selection marker genes (see FIG. 5), and then the offspring mice were mated with each other to obtain CCR8 humanized homozygous mice. The genotypes of the offspring mouse somatic cells were identified by PCR (primers are shown in Table 5). The identification result of an exemplary F1 generation mouse (with the Neo marker gene removed) is shown in FIGS. 6A-6D, where 7 F1 mice (F1-01, F1-02, F1-03, F1-06, F1-07, F1-08 and F1-09) were all identified as positive heterozygous mice. This indicates that the method of the instant application can be used to make a CCR8 humanized mouse without random insertion, wherein the genetic modification can be passed stably to the next generation.

TABLE 5
Primer sequence

| | | | Fragment |
|---|---|---|---|
| Primer | SEQ ID | Sequence(5′-3′) | size(bp) |
| WT-F | SEQ ID NO: 21 | ACACTAACACCCTGACATT | WT: 610 |
| | | GAGCCG | |
| WT-R | SEQ ID NO: 22 | GATGGTGGCTGTGACAGCA | |
| | | GCC | |
| WT-F | SEQ ID NO: 21 | ACACTAACACCCTGACATT | Mut: 236 |
| | | GAGCCG | |
| Mut-R | SEQ ID NO: 23 | CAGCAAGGAGCAACTTGCC | |
| | | ATTTG | |
| Frt-F | SEQ ID NO: 24 | TCAGCCTCCTGAACGCTGG | WT: 408 |
| | | ACCA | Mut: 502 |
| Frt-R | SEQ ID NO: 25 | TAGCTATCTTCAGACGCAC | (with Neo |
| | | CAG | being |
| | | | removed) |
| Flp-F | SEQ ID NO: 26 | GACAAGCGTTAGTAGGCAC | Mut: 325 |
| | | ATATAC | |
| Flp-R | SEQ ID NO: 27 | GCTCCAATTTCCCACAACA | |
| | | TTAGT | |

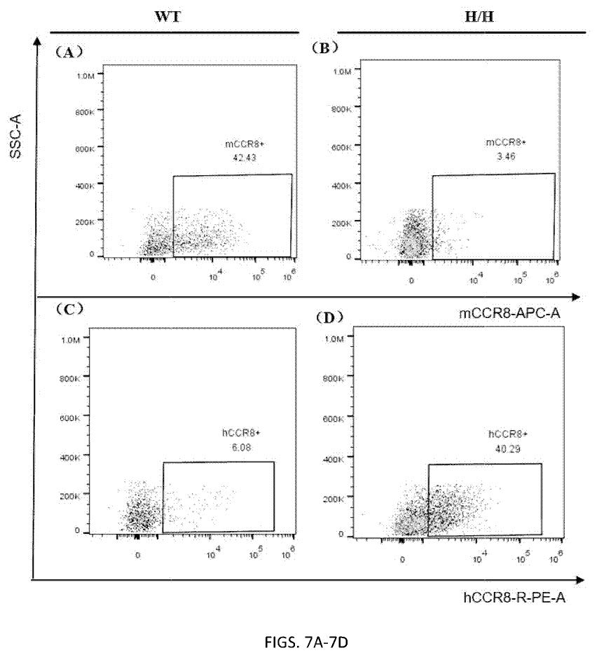

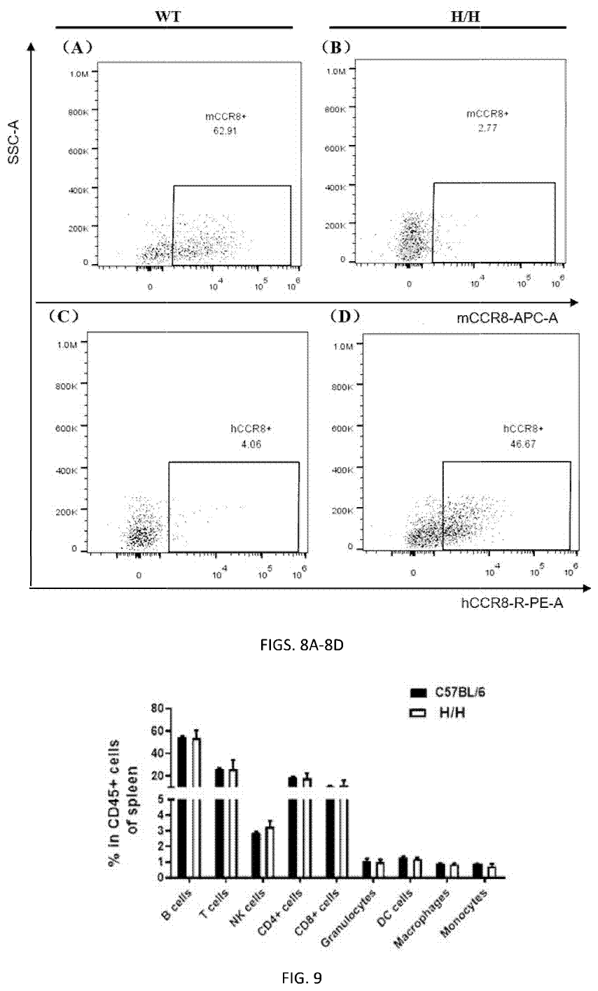

The expression of CCR8 in mouse tumors was detected by inoculating MC38 into CCR8 humanized homozygous mice. After the tumor reached about 200 mm³, the mice were enthanized. The tumor tissue was collected, subject to digestion, and then re-suspended into a single cell suspension for flow cytometry. Anti-mouse CD4 antibody anti-mCD4-BV421 (Brilliant Violet 421™ anti-mouse CD4), anti-mouse CCR8 antibody mCCR8-APC-A (APC anti-mouse CCR8 Antibody), anti-mFoxp3-PE/Cy7 (Anti-Mo/Rt Foxp3PE/Cy™ 7) and anti-human CCR8 antibody hCCR8-PE-A (PE anti-human CD198 (CCR8) Antibody) were used for staining in flow cytometry. The detection results are shown in FIGS. 7A-7D and FIGS. 8A-8D. As shown in FIGS. 7A-7D, mouse CCR8 protein can be detected in CD4+ T cells in wild-type C57BL/6 mice (FIG. 7A), but human CCR8 protein cannot be detected (FIG. 7C). In the CCR8 humanized homozygous mice, mouse CCR8 protein cannot be detected (FIG. 7B), but human CCR8 protein can be detected (FIG. 7D). As shown in FIGS. 8A-8D, mouse CCR8 protein can be detected in Treg cells in wild-type C57BL/6 mice (FIG. 8A), but human CCR8 protein cannot be detected (FIG. 8C). In the CCR8 humanized homozygous mice, mouse CCR8 protein cannot be detected (FIG. 8B), but human CCR8 protein can be detected (FIG. 8D).

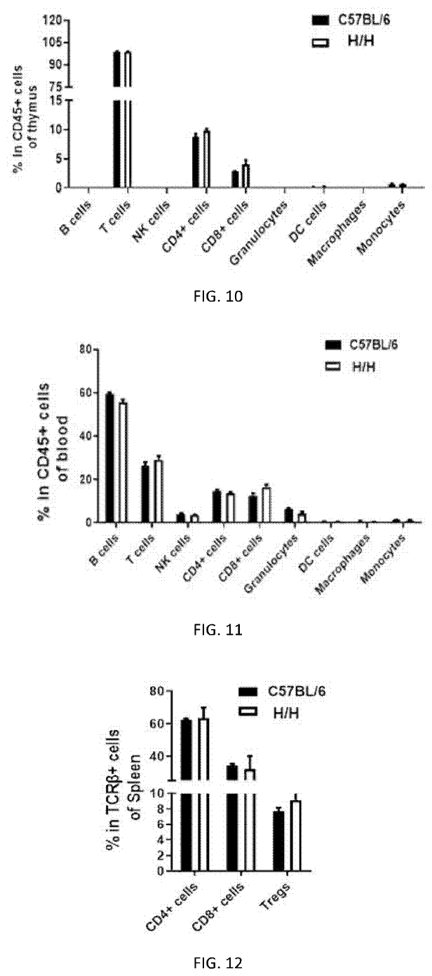

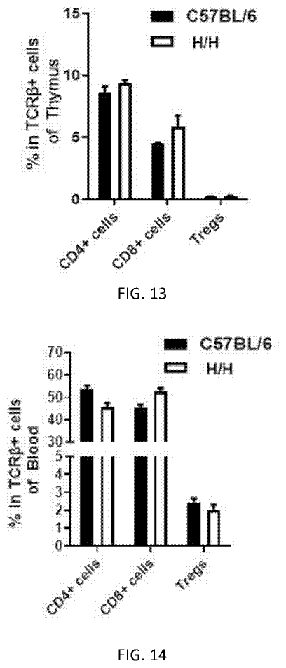

The immune cells of CCR8 humanized mice were further analyzed by flow cytometry. Specifically, 3 CCR8 humanized homozygous mice (H/H) and 3 wild-type C57BL/6 mice (7-8 weeks old) were selected, and immune cells and T cells were collected from the spleen, thymus and peripheral blood to perform T cell subpopulation analysis. As can be seen from FIGS. 9, 10, and 11, the leukocyte subpopulation profile in mice with humanized CCR8 gene is similar to that of wildtype C57BL/6 mice and shows no significant differences. Specifically, the percentages of B cells, T cells, NK cells, CD4+ cells (CD4+ cells), CD8+ cells (CD8+ cells), granulocytes (Granulocytes), DC cells (DC cells), macrophages, Monocytes, and T cell subtypes including CD4+ cells (CD4+ cells), CD8+ cells (CD8+ cells), and Treg cells (Tregs) in C57BL/6 mice and CCR8 humanized mouse were similar (FIGS. 12-14)). This indicates that the humanization of CCR8 does not affect the development of immune cells.

### Example 2. Preparation of Double Humanized or Multiple Humanized Mice

The CCR8 humanized mice prepared by this method can also be used to prepare double humanized or multi-humanized mouse models. For example, in the foregoing example 1, the embryonic stem cells used for blastocyst microinjection can be selected from mice containing genetic modifications on genes including CCR8, PD-1, PD-L1, CTLA4, B7H3, B7H4, CD47, IL2, IL23A, CCR2 and other genes. Alternatively, starting from the CCR8 humanized mice, isolation of mouse ES embryonic stem cells and gene recombination targeting technology can be used to obtain double-gene or multi-gene modified mouse models (with CCR8 and other genetic modifications). Alternatively, the homozygous or heterozygous CCR8 humanized mouse obtained by the instant method can also be mated with other to make genetically modified homozygous or heterozygous mice, and the offspring can be screened. According to Mendel's Laws of Heredity, there is a certain probability of obtaining double-gene or multi-gene modified humanized CCR8 mice, and then the heterozygotes can be mated with each other to obtain double-gene or multi-gene modified homozygotes. These double-gene or multi-gene modified mice can be used to verify the efficacy of drugs targeting human CCR8 and other gene regulators in vivo.

### Example 3. Immune Analysis of CCR8 Humanized Animal Model

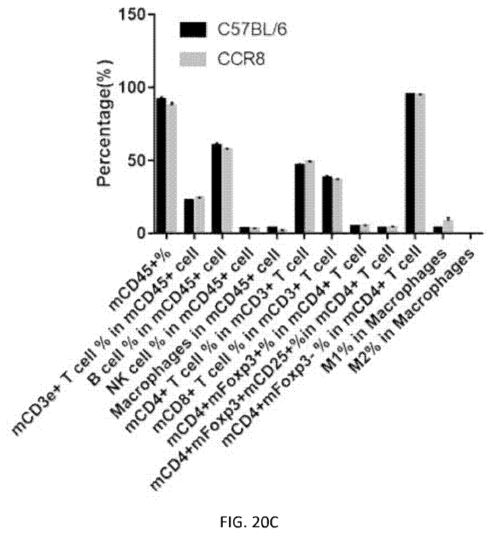

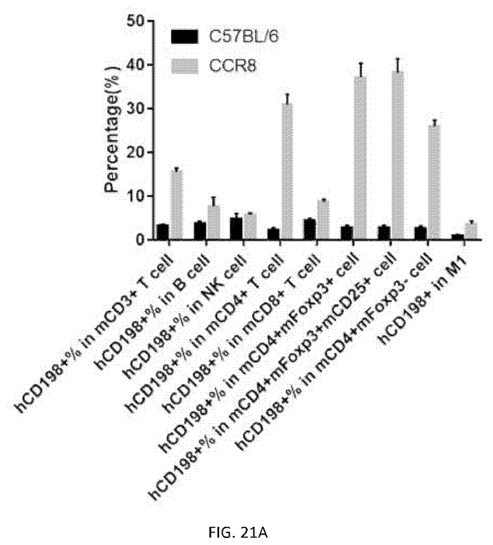

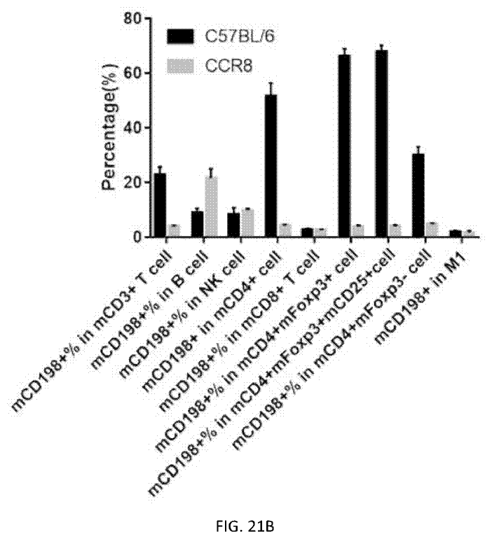

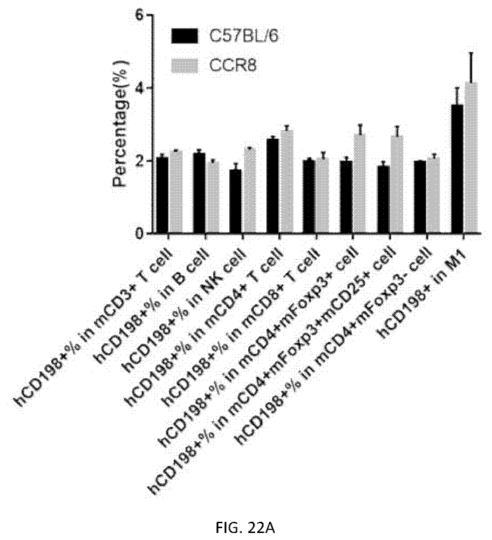

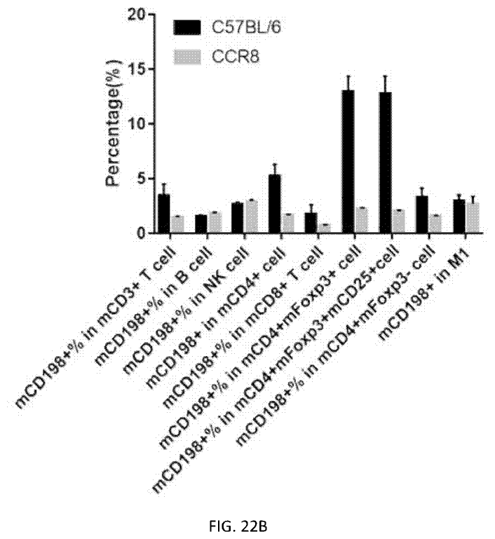

3 C57BL/6 mice and 3 CCR8 humanized homozygous mice (each of 8-9 weeks old) were selected, and subcutaneously inoculated with mouse colon cancer cell MC38. When the tumor reached about 1000 mm³, the mice were euthanized. Spleen cells, peripheral blood and tumors were collected. Anti-mouse CD45 antibody PerCP anti-mouse CD45 Antibody, anti-mouse TCR β antibody Brilliant Violet 711™ anti-mouse TCR β chain Antibody, anti-mouse CD4 antibody Brilliant Violet 510™ anti-mouse CD4 Antibody, anti-mouse CD8a antibody FITC anti-Mouse CD8a Antibody, Brilliant Violet 421™ anti-mouse NK1.1 Antibody, anti-mouse F4/80 antibody FITC anti-mouse F4/80 Antibody, anti-mouse CD3 antibody FITC anti-human CD3 Antibody, anti-mouse CD198 APC anti-mouse CD198 (CCR8) Antibody, anti-human CD198 antibody PE anti-human CD198 (CCR8) Antibody, and anti-mouse CD11b antibody PerCP anti-mouse/human CD11b Antibody were used for staining and flow cytometry was performed to detect the percentages of white blood cell subpopulations in spleen cells, peripheral blood and tumors. The results are shown in FIGS. 20A-20C. The percentages of mCD3ε T cells (mCD3e+ T cell), B cell (B cell), NK cell (NK cell) and macrophages (Macrophages) in mCD45 positive cells; the percentages of mCD4+ T cell (mCD4+ T cell), mCD8+ T cell (mCD8+ T cell), Treg cells (mCD4+mFoxp3+), mCD25+Treg cells (mCD4+mFoxp3+mCD25+) and non-Treg T cells (mCD4+mFoxp3−) in mCD4+ T cells; and M1 type macrophages (M1) and M2 type macrophages (M2) in macrophages were similar in the tumors (FIG. 20A), spleen cells (FIG. 20B) and peripheral blood (FIG. 20C) of homozygous CCR8 humanized mice and C57BL/6 mice.

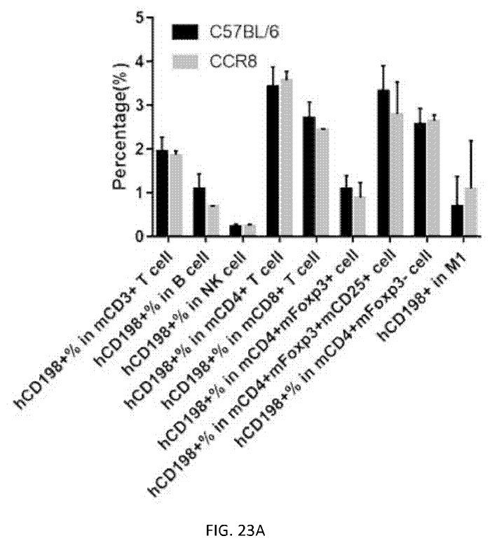

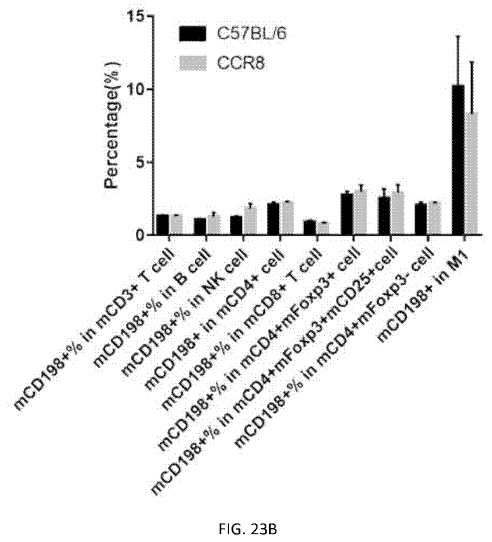

Furthermore, based on the above-mentioned flow cytometry data, the percentages of CCR8 (CD198) positive cells in leukocyte subtypes in spleen cells, peripheral blood and tumors was analyzed. The results are shown in FIGS. 21-23. The percentages of CCR8 is in CD3+ T cell (mCD3+ T cell), B cell (B cell), NK cell (NK cell), CD4+ T cell (mCD4+ T cell), CD8+ T cells (mCD8+ T cell), Treg cells (mCD4+mFoxp3+), mCD25+Treg cells (mCD4+mFoxp3+mCD25+), non-Treg T cells (mCD4+mFoxp3−) and M1 macrophages (M1) differed significantly in 1) CCR8 humanized homozygous mice and 2) C57BL/6 mouse tumor cells (FIGS. 21A-21B). In the tumor of humanized CCR8 homozygous mice, human CCR8 (hCD198) was highly expressed in Treg cells in the tumor, while mouse CCR8 (mCD198) was almost not expressed. In the tumor of C57BL/6 mice, mouse CCR8 was highly expressed in Treg cells, while human CCR8 was almost not expressed. In the spleen cells (FIGS. 22A-22B) and peripheral blood (FIGS. 23A-23B) of CCR8 humanized homozygous mice and C57BL/6 mice, CCR8 was almost not expressed in the above leukocyte subtypes. There was only limited CCR8 expression in the Treg cells of the spleen cells of C57BL/6 mice.

The above results indicate that the humanization of CCR8 gene does not affect the differentiation, development and distribution of leukocytes and T cells in mice in the spleen, lymphatic tissues and peripheral blood. Further, CCR8 is highly expressed on tumor-infiltrating Treg cells.

### Example 4. In Vivo Efficacy Verification Using the CCR8 Humanized Animal Model

CCR8 homozygous mice (5 weeks old) were selected, subcutaneously inoculated with mouse colon cancer cell MC38, and randomly divided into control group or treatment group (n=6/group) after the tumor size reached about 100 mm³. The treatment group was injected with the anti-human CCR8 antibody CCR8 Abl (10 mg/kg) (the sequence of CCR8 Abl is described in patent application WO20201384891A1). The control group was injected with human IgG1 (20 mg/kg). The frequency of administration is 2 times a week for a total of 6 times. The tumor volume was measured twice a week and the weight of the mice was measured. The mice were euthanatized after the tumor volume of a single mouse reached 3000 mm³. The specific drug or drug combination, dosage, administration route and frequency are shown in Table 6.

TABLE 6
drug, dosage, administration route and frequency

| Group | Drug | Dosage/Administration Route/Frequency |
|---|---|---|
| G1 | Human IgG1 | 20 mg/kg; Intraperitoneal (IP) Injection; |
| | | BIW; 6 doses in total |
| G2 | CCR8 Ab1 | 10 mg/kg; Intraperitoneal (IP) Injection; |
| | | BIW; 6 doses in total |

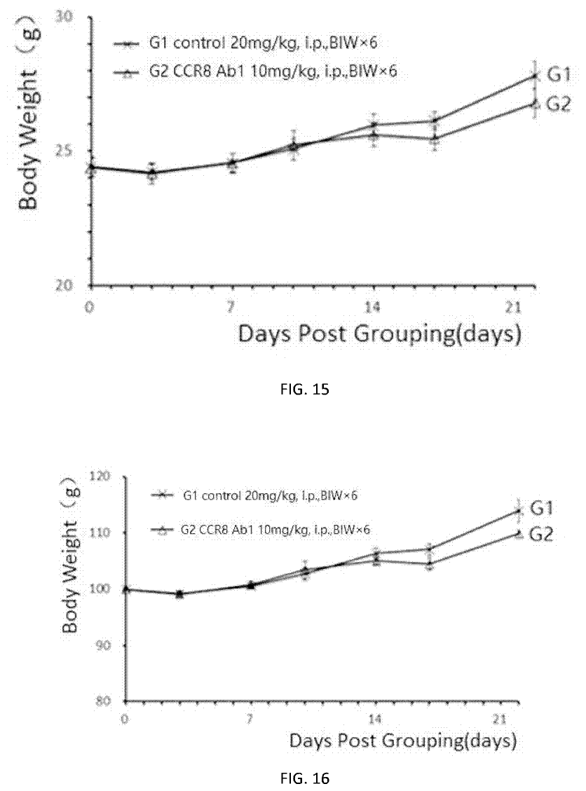

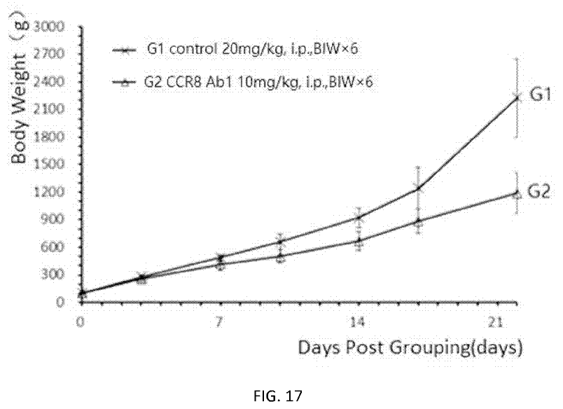

Overall, the animals in each group were in good health condition during the experiment. At the end of the experiment (the 22nd day after grouping), the body weight of mice in all treatment groups and control groups increased. There was no significant difference in body weight and body weight changes (P>0.05) (FIGS. 15 and 16). But from the results of tumor volume measurement (FIG. 17), while the tumor volume of the mice in the control group continued to grow during the experiment, tumor volume growth was suppressed and/or reduced in all the treatment groups compared with the control group. This indicates that the anti-CCR8 antibody had no obvious toxic effect on animals, had good safety profiles, and had tumor-inhibiting effects in vivo.

Table 7 lists the main data and analysis results of each experiment, including the tumor volume at the time of grouping and 10 days after grouping, the tumor volume at the end of the experiment (the 22nd day after grouping), the survival of the mice, the tumor (volume) inhibition rate (Tumor Growth Inhibition value, TGI_{TV}) and the statistical difference (P value) between the body weight and tumor volume of the mice in the treatment group and the control group.

TABLE 7
Tumor volume and tumor weight of mice in each group

| | | | | | | P value | |
|---|---|---|---|---|---|---|---|
| | Tumor volume(mm³) | | | | | Body | Tumor |
| | Day 0 | Day 10 | Day 22 | Survival | TGI_{TV} % | weight | volume |
| Control | 103 ± 5 | 656 ± 84 | 2226 ± 428 | 6/6 | N/A | N/A | N/A |
| Group G1 | | | | | | | |
| Treatment | 103 ± 5 | 501 ± 76 | 1191 ± 218 | 6/6 | 48.7 | 0.216 | 0.057 |
| Group G2 | | | | | | | |

It can be seen from Table 7 in combination with FIG. 15 and FIG. 16 that at the end of the experiment (day 22 after grouping), the body weight of the animals in each group increased and there was no significant difference in body weight (p>0.05), indicating that the animals tolerated the anti-CCR8 antibody well. From the results of tumor volume measurement (FIG. 17), the average tumor volume of the control group (G1) was 2226±428 mm³, the average tumor volume of the treatment group was 1191±218 mm³, and the tumor volume of the mice in the treatment group (G2) was reduced to different degrees comparing to the control group (G1), indicating that the anti-human CCR8 antibody can inhibit tumor growth, and can inhibit tumor growth in the humanized CCR8 mice (TGI_{TV}>48.7%).

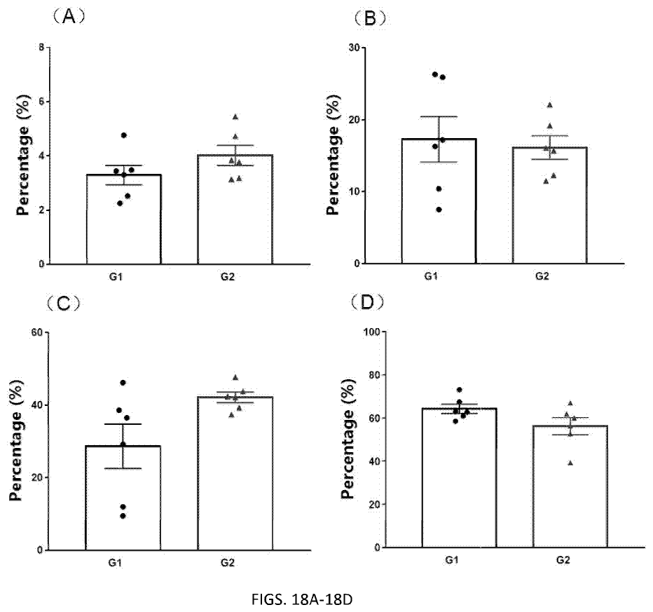

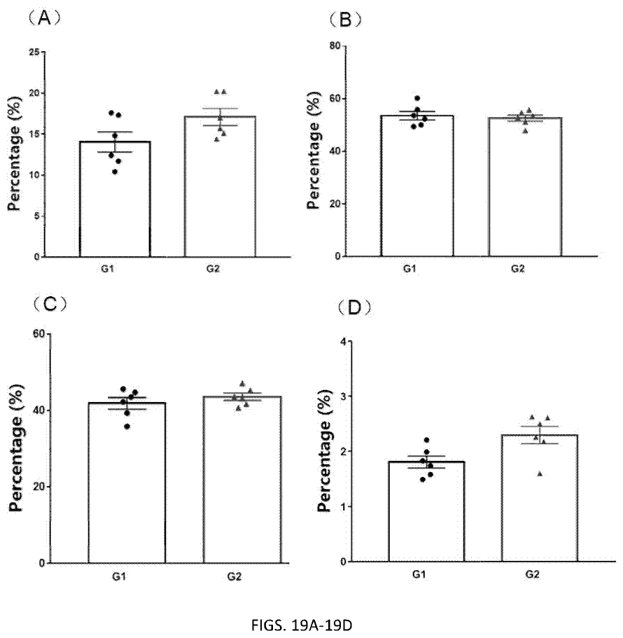

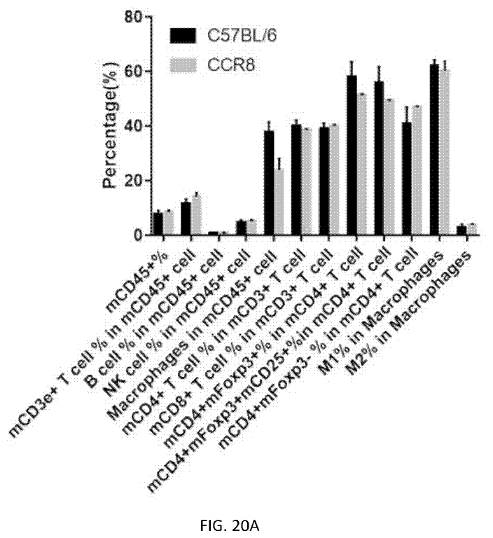

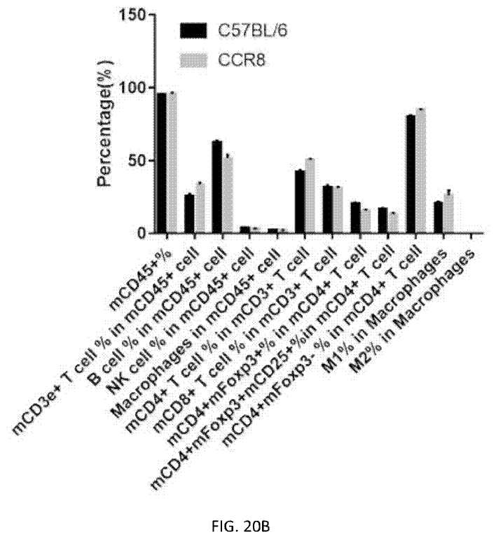

At the end of the experiment, the tumor and peripheral blood of mice in the control group G1 and treatment group G2 were collected, and the lymphocyte subpopulations in the peripheral blood and the lymphocyte subpopulations that infiltrated in the tumor were detected by flow cytometry, to detect the percentages of CD3+ cells were detected (mCD45+mCD3+), CD4+ cells (mCD45+mCD3+mCD4+mCD8−), CD8+ cells (mCD45+mCD3+mCD4−mCD8+), Treg cells (mCD45+mCD3+mCD4+mCD8−mFOXP3+). The test results are shown in FIGS. 18 and 19. In the tumor (FIG. 18) and peripheral blood (FIG. 19), the percentages of various types of cells in the G2 group was not significantly different from that in the G1 group.

The above research results show that the humanized CCR8 animal model can be used as a model for in vivo pharmacodynamic research, screening, evaluation and treatment experiments of CCR8 signaling pathway modulators, and can be used to evaluate the effectiveness of antibodies targeting human CCR8 in animals, and to evaluate the therapeutic effect of antibodies targeting CCR8.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric CCR8.

2. The animal of claim 1, wherein the sequence encoding the human or chimeric CCR8 is operably linked to an endogenous regulatory element at the endogenous CCR8 gene locus in the at least one chromosome.

3. The animal of claim 1 or 2, wherein the sequence encoding the human or chimeric CCR8 comprises a sequence encoding an amino acid sequence that is at least 70%, 75%, 80%, 85%, 90%, 95%, 99%, or 100% identical to SEQ ID NO: 2.

4. The animal of any one of claims 1-3, wherein the sequence encoding the human or chimeric CCR8 is operably linked to an endogenous 5′-UTR (e.g., immediately after 5′-UTR).

5. The animal of any one of claims 1-4, wherein the animal is a mammal, e.g., a monkey, a rodent or a mouse.

6. The animal of any one of claims 1-4, wherein the animal is a mouse or a rat.

7. The animal of any one of claims 1-6, wherein the animal does not express endogenous CCR8 or expresses a decreased level of endogenous CCR8 as compared to that of an animal without genetic modification.

8. The animal of any one of claims 1-7, wherein the animal has one or more cells expressing human or chimeric CCR8.

9. The animal of any one of claims 1-8, wherein the animal has one or more cells expressing human or chimeric CCR8, and human CCL1, human CCL8, human CCL16 or human CCL 18 can bind to the expressed human or chimeric CCR8.

10. The animal of any one of claims 1-8, wherein the animal has one or more cells expressing human or chimeric CCR8, and endogenous CCL1, endogenous CCL8, endogenous CCL16 or endogenous CCL 18 can bind to the expressed human or chimeric CCR8.

11. A genetically-modified, non-human animal, wherein the genome of the animal comprises a replacement of a sequence encoding a region of endogenous CCR8 with a sequence encoding a corresponding region of human CCR8 at an endogenous CCR8 gene locus.

12. The animal of claim 11, wherein the sequence after the replacement is operably linked to an endogenous regulatory element at the endogenous CCR8 locus, and one or more cells of the animal express human CCR8 or chimeric CCR8.

13. The animal of claim 11 or 12, wherein the animal does not express endogenous CCR8 or expresses a decreased level of endogenous CCR8 as compared to that of an animal without genetic modification.

14. The animal of any one of claims 11-13, wherein the replaced region is located immediately after 5′-UTR at the endogenous CCR8 locus.

15. The animal of any one of claims 11-14, wherein the animal has one or more cells expressing a chimeric CCR8 having one or more humanized extracellular regions, transmembrane regions, and cytoplasmic regions, wherein one or more of the humanized extracellular regions comprise a sequence that is at least 50%, 60%, 70%, 80%, 90%, 95%, or 99% identical to the corresponding extracellular region of human CCR8.

16. The animal of any one of claims 11-15, wherein one or more of the humanized extracellular regions of the chimeric CCR8 has a sequence that has at least 10 contiguous amino acids that are identical to a contiguous sequence present in the corresponding extracellular region of human CCR8.

17. The animal of any one of claims 11-16, wherein the animal is a mouse, and the replaced region comprises or consists of the entirety or a portion of the coding sequence in exon 2 of the endogenous mouse CCR8 gene.

18. The animal of any one of claims 11-17, wherein the animal is heterozygous with respect to the replacement at the endogenous CCR8 gene locus.

19. The animal of any one of claims 11-17, wherein the animal is homozygous with respect to the replacement at the endogenous CCR8 gene locus.

20. A method for making a genetically-modified, non-human animal, comprising:
replacing in at least one cell of the animal, at an endogenous CCR8 gene locus, a sequence encoding a region of an endogenous CCR8 with a sequence encoding a corresponding region of human CCR8 gene.

21. The method of claim 20, wherein the sequence encoding the corresponding region of human CCR8 gene comprises exon 2, or a part thereof, of a human CCR8 gene.

22. The method of claim 20 or 21, wherein the sequence encoding a corresponding region of human CCR8 gene encodes a sequence that is at least 90% identical to SEQ ID NO: 2.

23. The method of any one of claims 20-22, wherein the animal is a mouse, and the endogenous CCR8 locus is exon 2 of the mouse CCR8 gene.

24. The method of any one of claims 20-23, wherein the replaced region is located in exon 2 of the mouse CCR8 gene.

25. A non-human animal comprising at least one cell comprising a nucleotide sequence encoding a humanized CCR8 polypeptide, wherein the humanized CCR8 polypeptide comprises at least 50 contiguous amino acid residues that are identical to the corresponding contiguous amino acid sequence of a human CCR8, wherein the animal expresses the humanized CCR8.

26. The animal of claim 25, wherein the humanized CCR8 polypeptide has at least 10 contiguous amino acid residues that are identical to the corresponding contiguous amino acid sequence of a human CCR8 extracellular region.

27. The animal of claim 25, wherein the humanized CCR8 polypeptide comprises a sequence that is at least 90%, 95%, or 99% identical to SEQ ID NO: 2.

28. The animal of any one of claims 25-27, wherein the nucleotide sequence is operably linked to an endogenous CCR8 regulatory element of the animal (e.g., 5′-UTR).

29. The animal of any one of claims 25-28, wherein the humanized CCR8 polypeptide comprises one or more humanized extracellular region, one or more humanized CCR8 transmembrane region and/or one or more humanized CCR8 cytoplasmic region.

30. The animal of any one of claims 25-29, wherein the nucleotide sequence is integrated to an endogenous CCR8 gene locus of the animal.

31. A method of making a genetically-modified mouse cell that expresses a human CCR8 or a chimeric CCR8, the method comprising:
replacing at an endogenous mouse CCR8 gene locus, a nucleotide sequence encoding a region of mouse CCR8 with a nucleotide sequence encoding a corresponding region of human CCR8, thereby generating a genetically-modified mouse cell that includes a nucleotide sequence that encodes the human CCR8 or the chimeric CCR8, wherein the mouse cell expresses the human CCR8 or the chimeric CCR8.

32. The method of claim 31, wherein the entire coding sequence of mouse CCR8 gene is replaced by the entire coding sequence of human CCR8 gene.

33. The method of claim 31 wherein the chimeric CCR8 comprises:
one or more of the extracellular regions of human CCR8; and
one or more of the transmembrane regions; and/or one or more of the cytoplasmic regions of mouse CCR8.

34. The animal of any one of claims 1-19 and 25-30, wherein the animal further comprises a sequence encoding an additional human or chimeric protein.

35. The animal of claim 34, wherein the additional human or chimeric protein is cytotoxic T-lymphocyte-associated protein 4 (CTLA-4), Lymphocyte Activating 3 (LAG-3), B And T Lymphocyte Associated (BTLA), Programmed cell death protein 1 (PD-1), Programmed death-ligand 1 (PD-L1), CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), or TNF Receptor Superfamily Member 4 (OX40).

36. The method of any one of claims 20-24 and 31-33, wherein the animal or mouse further comprises a sequence encoding an additional human or chimeric protein.

37. The method of claim 36, wherein the additional human or chimeric protein is CTLA-4, LAG-3, BTLA, PD-1, PD-L1, CD27, CD28, CD40, CD47, CD137, CD154, TIGIT, TIM-3, GITR, or OX40.

38. A method of determining effectiveness of an anti-CCR8 antibody for the treatment of cancer, comprising:
administering the anti-CCR8 antibody to the animal of any one of claims 1-19 and 25-30, wherein the animal has a tumor; and
determining the inhibitory effects of the anti-CCR8 antibody to the tumor.

39. The method of claim 38, wherein the tumor comprises one or more cells that express a CCR8 ligand.

40. The method of claim 38, wherein the tumor comprises one or more cancer cells that are injected into the animal.

41. The method of claim 38, wherein determining the inhibitory effects of the anti-CCR8 antibody to the tumor comprises measuring the tumor volume in the animal.

42. The method of claim 38, wherein the tumor cells are breast cancer cells, colon cancer cells, or lung cancer cells.

43. A method of determining effectiveness of an anti-CCR8 antibody and an additional therapeutic agent for the treatment of a tumor, comprising
administering the anti-CCR8 antibody and the additional therapeutic agent to the animal of any one of claims 1-19 and 25-30, wherein the animal has a tumor; and
determining the inhibitory effects on the tumor.

44. The method of claim 43, wherein the animal further comprises a sequence encoding a human or chimeric PD-1.

45. The method of claim 43, wherein the additional therapeutic agent is an anti-PD-1 antibody.

46. The method of claim 43, wherein the tumor is caused by injection of one or more cancer cells into the animal.

47. The method of claim 43, wherein determining the inhibitory effects of the treatment involves measuring the tumor volume in the animal.

48. The method of claim 43, wherein the animal has breast cancer, colon cancer, or lung cancer.

49. A protein comprising an amino acid sequence, wherein the amino acid sequence is one of the following:
(a) an amino acid sequence set forth in SEQ ID NO: 2;
(b) an amino acid sequence that is at least 90% identical to SEQ ID NO: 2;
(c) an amino acid sequence that is at least 91%, 92%, 93%, 94%, 95%, 96%, 97%, 98%, or 99% identical to SEQ ID NO: 2;
(d) an amino acid sequence that is different from the amino acid sequence set forth in SEQ ID NO: 2 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 2.

50. A nucleic acid comprising a nucleotide sequence, wherein the nucleotide sequence is one of the following:
(a) a sequence that encodes the protein of claim 50;
(b) SEQ ID NO: 3
(c) SEQ ID NO: 4
(d) SEQ ID NO: 5;
(e) SEQ ID NO: 6;
(f) SEQ ID NO: 7;
(g) SEQ ID NO: 8;
(h) SEQ ID NO: 9;
(i) SEQ ID NO: 10;
(j) a sequence that is at least 90% identical to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10;
(k) a sequence that is at least 91%, 92%, 93%, 94%, 95%, 96%, 97%, 98%, or 99% identical to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

51. A cell comprising the protein of claim 49 and/or the nucleic acid of claim 50.

52. An animal comprising the protein of claim 49 and/or the nucleic acid of claim 50.
